(19) United States
(12) Patent Application Publication
(10) Pub. No.: US 2013/0158578 A1
(43) Pub. Date: Jun. 20, 2013

(54) NEUROSURGICAL DEVICES AND ASSOCIATED SYSTEMS AND METHODS

(71) Applicants: Basavaraj Ghodke, Mercer Island, WA (US); Daniel Cooke, San Francisco, CA (US); Robert Wilcox, Bothell, WA (US)

(72) Inventors: Basavaraj Ghodke, Mercer Island, WA (US); Daniel Cooke, San Francisco, CA (US); Robert Wilcox, Bothell, WA (US)

(73) Assignee: UNIVERSITY OF WASHINGTON, Seattle, WA (US)

(21) Appl. No.: 13/820,739

(22) PCT Filed: Sep. 2, 2011
(86) PCT No.: PCT/US11/50443

§ 371 Date: Mar. 4, 2013

Related U.S. Application Data
(60) Provisional application No. 61/380,030, filed on Sep. 3, 2010.

(51) Int. Cl. A61B 17/32

(52) U.S. Cl. CPC A61B 17/320016
USPC 606/170

(57) ABSTRACT

Neurosurgical devices including or used with cannulas or catheters and associated systems and methods are disclosed herein. The neurosurgical devices can include, for example, a cannula having a main portion and an angle-forming member proximate a distal end of the main portion. The angle-forming member can be configured to transition from a substantially straight configuration while the cannula is advanced through tissue along a substantially straight first portion of a path to an angled configuration when the angle-forming member reaches an end of the substantially straight first portion of the path. The neurosurgical devices also can include, for example, a neurosurgical catheter including a surface disrupter, an elongated macerator, or a lateral opening. Neurosurgical catheterization portals also are disclosed. The neurosurgical catheterization portals can, for example, have an adjustable portal that is movable relative to a body to accommodate different entry angles of a catheterization path.

### CROSS-REFERENCE TO RELATED APPLICATION(S)

This application claims the benefit of pending U.S. Provisional Patent Application No. 61/380,030, entitled “SYSTEMS AND METHODS FOR RAPID INTRACRANIAL EVACUATION,” filed Sep. 3, 2010, which is incorporated herein by reference in its entirety.

### TECHNICAL FIELD

The present technology relates generally to neurosurgery. In particular, several embodiments are directed to neurosurgical devices including or used with cannulas or catheters and associated systems and methods.

### BACKGROUND

Neurosurgery, which includes surgical procedures performed on any portion of the central nervous system (CNS), can be useful for the treatment of a variety of conditions, such as brain cancer, hydrocephalus, stroke, aneurysm, and epilepsy. The complexity and fragility of the CNS, however, make surgical treatment of the CNS more challenging than surgical treatment of other body systems. Tumors and other pathologies can occur in portions of the CNS that are effectively inaccessible to surgery. Such inaccessibility can occur, for example, when the pathologies are located within or proximate to eloquent portions of the brain, i.e., portions of the brain that control essential functions, such as movement and speech. Even minor disturbance of structures within eloquent portions of the brain can irreparably damage the brain's functionality.

The risk of infection is especially severe in neurosurgical procedures. Rather than relying on the immune system, the CNS is adapted to avoid infection primarily by isolation. Surrounding structures protect the CNS from pathogens outside the body. The blood-brain barrier protects the CNS from most pathogens inside the body. With few exceptions, the blood-brain barrier prevents bacteria in the bloodstream from entering the CNS. Neurosurgical procedures typically include a craniotomy in which a bone flap is temporarily removed from the skull to access the brain. A craniotomy compromises the isolation of the CNS and exposes the brain to the potential introduction of external pathogens. Bacteria entering the site of a craniotomy can cause a serious brain infection leading, for example, to meningitis or abscess. Such infections can be particularly difficult to treat, in part, because the blood-brain barrier tends to exclude antibiotics.

To a greater degree than most types of surgery, neurosurgery achieves better results when it is minimally invasive and extremely precise. Detailed planning is common in neurosurgery. During planning, a neurosurgeon typically reviews images and other data related to CNS morphology and physiology, which can vary considerably between patients. Imaging (e.g., computed tomography (CT) and magnetic resonance imaging (MRI)) can be used to develop a map of a portion of the CNS (e.g., a portion of the brain) from which a path to an area targeted for neurosurgical intervention can be formulated. During neurosurgery, imaging can be used to navigate instruments and monitor the status of affected tissue. Due to the imaging requirements and the need for extra precautions to prevent infection, a full surgical theater is currently used for most neurosurgical procedures.

The high cost and potential complications of conventional neurosurgery typically make it a treatment of last resort. Currently, neurosurgery is rarely used for the treatment of emergency conditions, despite its potential utility. Some types of stroke, for example, would benefit from immediate neurosurgical intervention. A stroke occurs when the blood supply to the brain is disrupted. The length of time prior to correcting the cause of the disruption can be the primary determinant of the condition's outcome. The short window of opportunity for treatment can make it difficult to complete the surgical planning and other preparation involved in conventional neurosurgery. Furthermore, most conventional neurosurgical devices, systems, and methods are designed for non-emergency applications.

### BRIEF DESCRIPTION OF THE DRAWINGS

FIGS. 1A-1C are schematic views of a catheterization system at different stages of deployment into brain tissue in accordance with an embodiment of the present technology.

FIGS. 2A-2D are schematic views of a catheterization system at different stages of deployment into brain tissue in accordance with an embodiment of the present technology.

FIG. 3 is a perspective view of a skull mount configured in accordance with an embodiment of the present technology.

FIG. 4 is an exploded perspective view of the skull mount of FIG. 3.

FIG. 5 is a perspective view of a base of the skull mount of FIG. 3.

FIG. 6 is a cross-sectional view of the base of the skull mount of FIG. 3.

FIG. 7 is a perspective view of a cap of the skull mount of FIG. 3.

FIG. 8 is a cross-sectional view of the cap of the skull mount of FIG. 3.

FIG. 9 is a schematic view of a catheter distal portion configured in accordance with an embodiment of the present technology.

FIG. 10A is a schematic view of a catheter distal portion configured in accordance with an embodiment of the present technology.

FIG. 10B is a perspective view of the catheter distal portion of FIG. 10A.

FIG. 11 is a schematic view of a catheter distal portion configured in accordance with an embodiment of the present technology.

FIG. 12A is a schematic view of a catheter distal portion configured in accordance with an embodiment of the present technology with a surface disrupter in a collapsed configuration.

FIG. 12B is a schematic view of the catheter distal portion of FIG. 12A with the surface disrupter in an expanded configuration.

FIG. 13 is a schematic view of a catheter distal portion configured in accordance with an embodiment of the present technology.

FIG. 14 is a schematic view of a catheter distal portion configured in accordance with an embodiment of the present technology.

FIG. 15 is a schematic view of a catheter distal portion configured in accordance with an embodiment of the present technology.

FIG. 16A is a perspective view of a catheter distal portion configured in accordance with an embodiment of the present technology.

FIG. 16B is an exploded perspective view of a suction conduit of the catheter distal portion of FIG. 16A.

FIG. 17 is a schematic view of a catheter distal portion configured in accordance with an embodiment of the present technology.

FIG. 18 is a schematic view of a catheter controller configured in accordance with an embodiment of the present technology.

FIG. 19 is a schematic view of a catheter distal portion configured in accordance with an embodiment of the present technology.

FIG. 20 is a perspective view of an ultrasound transducer configured in accordance with an embodiment of the present technology.

FIG. 21 is a block diagram of an ultrasonography system configured in accordance with an embodiment of the present technology.

### DETAILED DESCRIPTION

The present technology is directed to devices, systems, and methods related to neurosurgery, such as neurosurgery including transcranial catheterization. Several embodiments of the present technology can be used for a variety of neurosurgical applications, such as neurosurgical applications involving both linear and nonlinear access to various portions of the CNS, including subcortical portions of the brain, with minimal damage to eloquent tissue. For example, several embodiments are well suited for removing material from the brain, such as tumors, intraparenchymal clots, and intraventricular clots. Several of these embodiments can allow for the removal of clots that a conventional thrombolytic therapy cannot evacuate. Several embodiments of the present technology can be well suited for the removal of a discrete volume of target tissue while preventing the removal of non-target tissue, especially when both tissues have similar material properties, such as with clot and brain tissue. Several embodiments of the present technology can also be well suited for the implantation or delivery of brain-stimulating electrodes (e.g., wire electrodes), radiofrequency devices, extravascular stents, shunts, cells (e.g., stem cells), drugs, and drug reservoirs. In addition, treatments administered in accordance with several embodiment of the present technology can provide therapeutic benefits without removing material from the CNS or delivering material to the CNS. For example, such treatments can be used to provide cooling, heating, or electrical stimulation to portions of the CNS.

Several embodiments of the present technology are expected to provide superior treatments for a variety of conditions, often at lower cost than conventional therapies. For example, significantly improved outcomes are expected relative to current protocols for the treatment of deep intracerebral hemorrhage. Current protocols for the treatment of deep intracerebral hemorrhage involve the use of a ventriculostomy catheter in concert with chemical thrombolysis, which can take hours to days to reduce the hemorrhagic volume and its associated mass effect. Such treatment often requires the use of an operative theater at a higher cost than that of a biplane fluoroscopy suit. In addition, the neuro-navigational software used in the treatment of deep intracerebral hemorrhage according to current protocols typically provides a virtual representation of the practitioner's instrument and thus cannot account for anatomical changes that occur as the brain is manipulated and the hemorrhage removed. In contrast, several embodiments of the present technology can be used to perform a mechanical thrombectomy in acute stroke intervention. In comparison with conventional treatments, treatments in accordance with several embodiments of the present technology are expected to permit faster and more substantial hemorrhage removal with less damage to surrounding structures. In addition to or instead of stoke, several embodiments of the present technology can be used for diagnosis and treatment of other head, neck, and CNS pathologies, such as brain tumors, aneurysm, hydrocephalus, abscess, neurodegenerative disorders, vascular anomalies, and epilepsy.

The following description provides many specific details for a thorough understanding of, and enabling description for, embodiments of the present technology. Well-known structures and systems as well as methods often associated with such structures and systems have not been shown or described in detail to avoid unnecessarily obscuring the description of the various embodiments of the disclosure. In addition, those of ordinary skill in the relevant art will understand that additional embodiments can be practiced without several of the details described below.

Throughout this disclosure, the singular terms “a,” “an,” and “the” include plural referents unless the context clearly indicates otherwise. Similarly, the word “or” is intended to include “and” unless the context clearly indicates otherwise. Directional terms, such as “upper,” “lower,” “front,” “back,” “vertical,” and “horizontal,” may be used herein to express and clarify the relationship between various elements. It should be understood that such terms do not denote absolute orientation.

### 1. Constrained Deployment

Conventional catheterization is typically used for vascular applications, e.g., for angioplasty. In vascular applications, the vasculature defines the catheterization path within the body. To travel within the vasculature, a catheter typically must be flexible and bend gradually as the vessels bend. Steerable catheters can be used to navigate through branching vessels as needed to reach a target. Unlike vascular applications, catheterization of CNS tissue typically proceeds without a defined anatomical path. As a result, conventional approaches to catheterization of CNS tissue often are limited to use of a straight path through a rigid cannula. This is inadequate when a target portion of the CNS cannot be accessed without navigating around eloquent tissue and brain structures via a nonlinear path.

Neurosurgical catheterization in accordance with several embodiments of the present technology can include introducing a cannula or catheter into CNS tissue to define a linear path or a nonlinear path to a target area. A nonlinear path, for example, can include two or more substantially straight portions and an angle between each of the substantially straight portions. The path can have varying levels of complexity according to the position of a target area relative to eloquent portions of the CNS. Devices and systems configured in accordance with several embodiments of the present technology can be capable of forming complex paths, including paths that extend through portions of the ventricular space of the brain to reach a target area. Movement within the ventricular space of the brain typically is less likely to damage eloquent tissue than movement through other portions of the brain. Some paths extend through a non-eloquent portion of the cortex, into the ventricular space of the brain, through the ventricular space, and then back into the cortex to reach a target area. Devices and systems configured in accordance with several embodiments of the present technology can be configured such that the path is formed without substantially disturbing tissue around the path. This objective typically does not apply to vascular catheterization. Blood vessels are flexible and movable within surrounding material, so simply pushing and twisting a vascular cannula or catheter can cause it to advance with no detrimental effect. In contrast, any movement of an object through CNS tissue can permanently damage the tissue. In neurosurgical catheterization, damage to tissue directly along a single path is unavoidable. Damage to tissue around that path, however, can be substantially avoided using several embodiments of the present technology.

Forming a path including an angle using a structure substantially constrained to the path is technically challenging. For example, conventional approaches, such as laterally shifting a cannula while the cannula is deployed or advancing a bent cannula through tissue, would disturb CNS tissue surrounding the path. Devices and systems configured in accordance with several embodiments of the present technology include articulated or telescoping elements that can advance along a path without substantially disturbing tissue surrounding the path. For example, such embodiments can include an angle-forming member that transitions from being substantially straight while passing along a substantially straight portion of a path to being angled when positioned at a portion of the path where a change of direction is desired. After the angle is formed, the angle-forming member can remain substantially stationary within the CNS tissue. Further advance along the path can include sliding a separate structure within or around the angle of the angle-forming member.

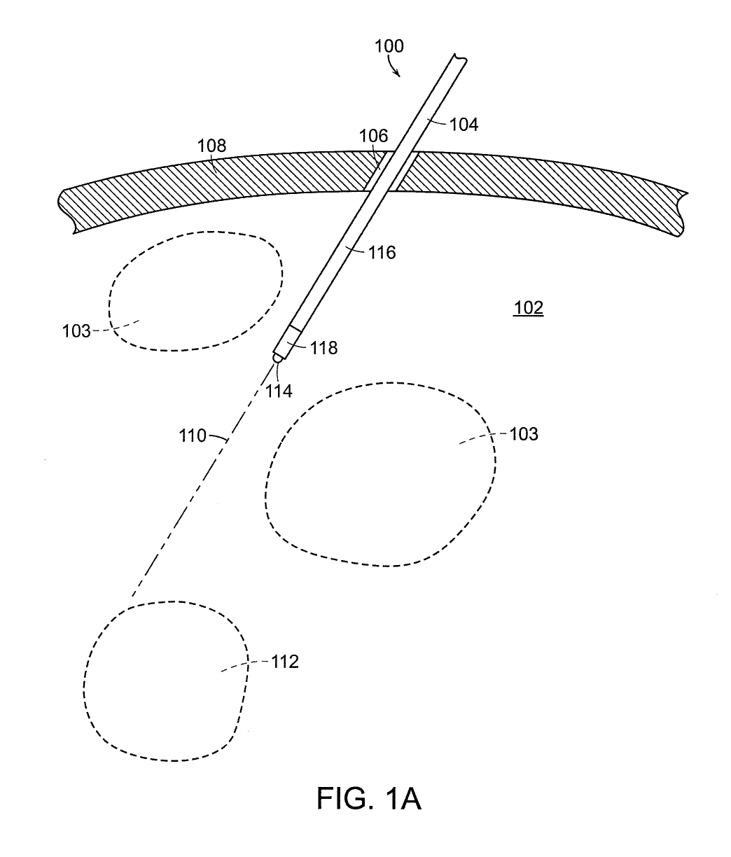

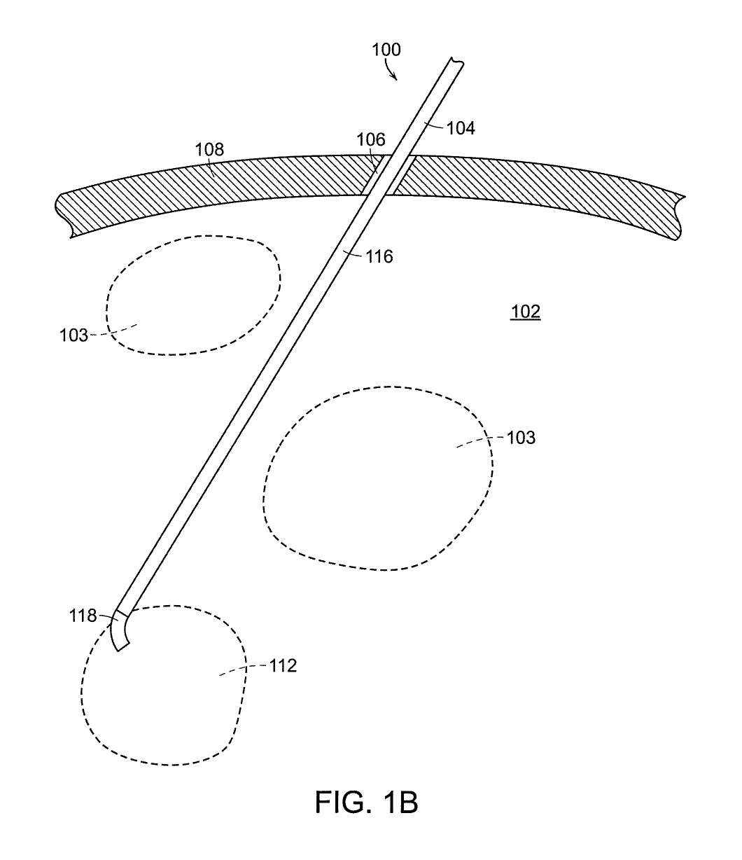

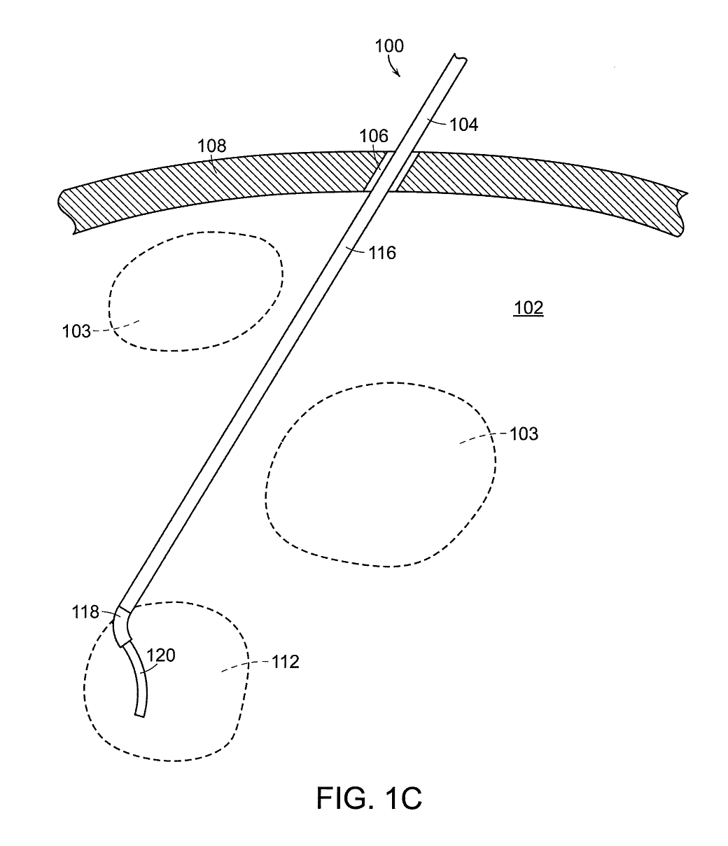

FIGS. 1A-1C illustrate a catheterization system 100 configured in accordance with an embodiment of the present technology during deployment into brain tissue 102 having eloquent portions 103. As shown in FIG. 1A, the catheterization system 100 includes a cannula 104, that is inserted into the brain tissue 102 through an opening 106 in a skull 108 and advanced along a path 110 through the brain tissue to a target area 112. An obturator 114 can be used to facilitate advancement of the cannula 104 along the path 110. The obturator 114, for example, can be positioned within the cannula 104 with a rounded tip of the obturator protruding slightly beyond a distal end of the cannula. The rounded tip can serve to dissect brain tissue 102 as the obturator 114 and the cannula 104 are advanced. Alternatively, instead of an obturator 114, other portions of the catheterization system 100 can be positioned within the cannula 104 as it is advanced. For example, a catheter (not shown) having a distal end suitable for dissecting the brain tissue 102 can take the place of the obturator 114.

The cannula 104 includes a straight portion 116 and an angle-forming member 118 at its distal end. The straight portion 116 is substantially rigid. Since the path 110 through the brain tissue 102 is unconstrained, the rigid structure of the straight portion 116 of the cannula 104 can help to keep other portions of the catheterization system 100 in position. In several embodiments of the present technology, a rigid portion of a cannula, such as the straight portion 116 of the cannula 104 is constrained within a catheterization portal fixedly attached to a patient's skull. For example, the straight portion 116 of the cannula 104 can be slidingly received snugly within a rigid sleeve of a catheterization portal. Axial mobility of the straight portion 116 of the cannula 104 can be suspended after the straight portion is positioned in the brain tissue 102. For example, catheterization portals configured in accordance with several embodiments of the present technology can include locking mechanisms, such as pressure screws, configured to engage a side wall of the straight portion 116 of the cannula 104 after the straight portion is positioned in the brain tissue 102. Additional details regarding catheterization portals configured in accordance with several embodiments of the present technology are provided below.

The length of the angle-forming member 118 is much smaller than the length of the straight portion 116. In several embodiments of the present technology, the angle-forming member 118 has a length between about 2 times and about 15 times its diameter, such as between about 3 times and about 10 times its diameter. In other embodiments, however, the angle-forming member 118 can have a different configuration. While advancing along the path 110, the angle-forming member 118 remains substantially straight. As shown in FIG. 1B, when the target area 112 is reached, the angle-forming member 118 is actuated to form a compact angle. This actuation can occur according to one of several mechanisms. In the illustrated catheterization system 100, the angle-forming member 118 includes a spring pre-tensioned at a desired angle and then encapsulated in a flexible polymer. While the obturator 114, which is substantially rigid, is positioned within the angle-forming member 118, the angle-forming member is forced into a substantially straight configuration.

As shown in FIG. 1B, upon reaching the target area 112, the cannula 104 can be advanced past the obturator 114, which allows the angle-forming member 118 to regain its relaxed configuration. The obturator 114 also can be partially or fully withdrawn to cause the angle-forming member 118 to regain its relaxed configuration. An angle-forming member 118 having any desired pre-tensioned angle for executing a particular neurosurgical plan can be loaded onto the distal end of the straight portion 116 of the cannula 104 prior to a procedure. Alternatively, a neurosurgical kit configured in accordance with several embodiments of the present technology can include a set of cannulas 104 having angle-forming members 118 with different pre-tensioned angles (e.g., 15°, 30°, and 45°). A neurosurgeon can select an appropriate cannula 104 from the set of cannulas for executing a particular neurosurgical plan.

As shown in FIG. 1C, after the angle-forming member 118 regains its relaxed configuration, the obturator 114 is fully withdrawn. A catheter 120 is inserted into the cannula 104 in place of the obturator 114. The catheter 120 has significant mobility within the target area. The catheter 120 exits the cannula 104 at a defined angle of the angle-forming member 118, is rotatable, and is steerable in a serpentine manner, such as according a steering mechanism known in the art for vascular catheterization. Within the target area 112, limiting movement to a single path can be less important than outside the target area. The catheter 120, therefore, can be moved through intermediate positions as needed to execute a desired treatment of the target area 112. As described below, other embodiments of the present technology can include different catheter configurations, including catheters with two or more articulations and joints.

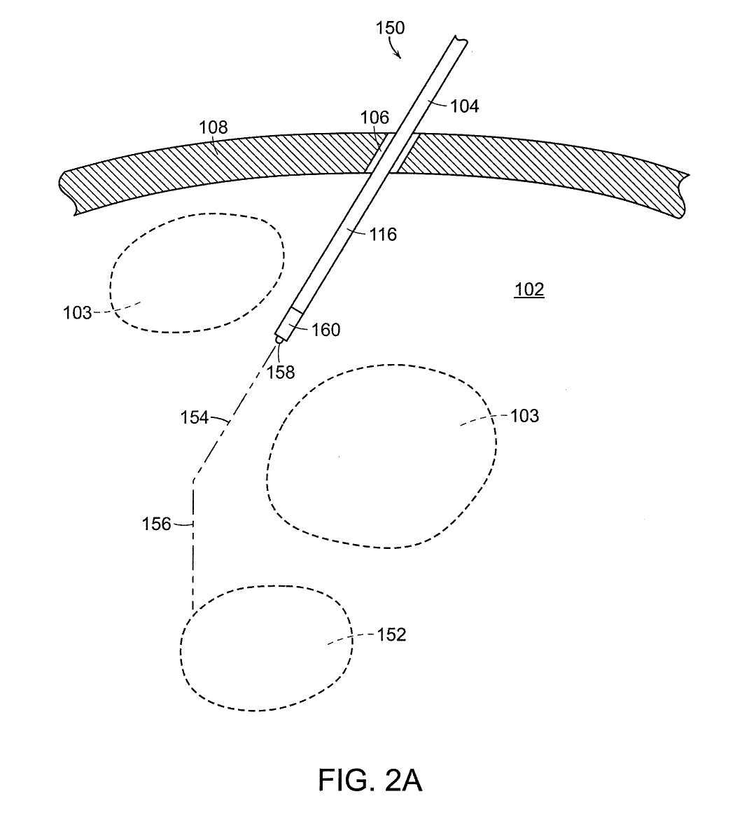

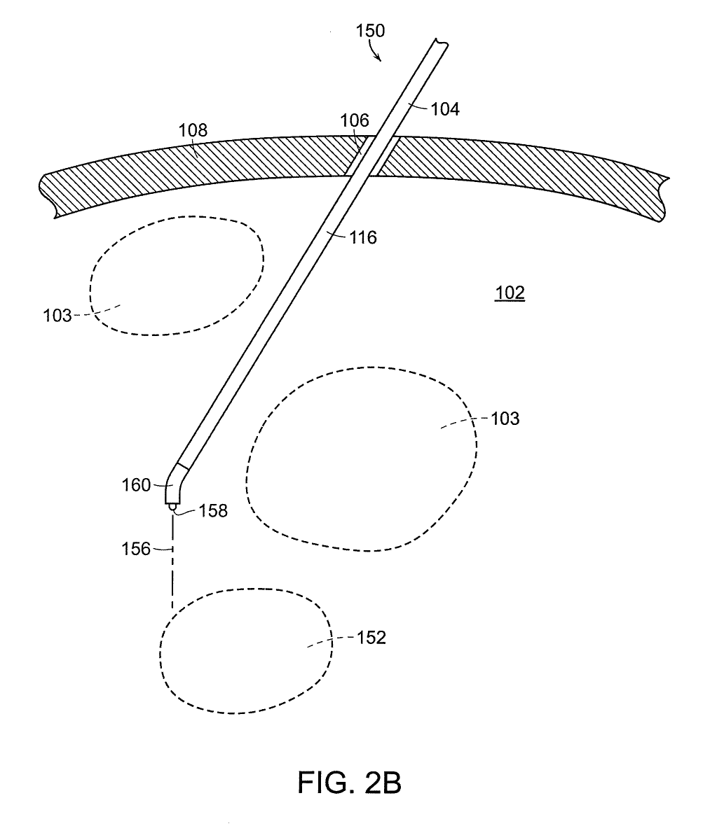

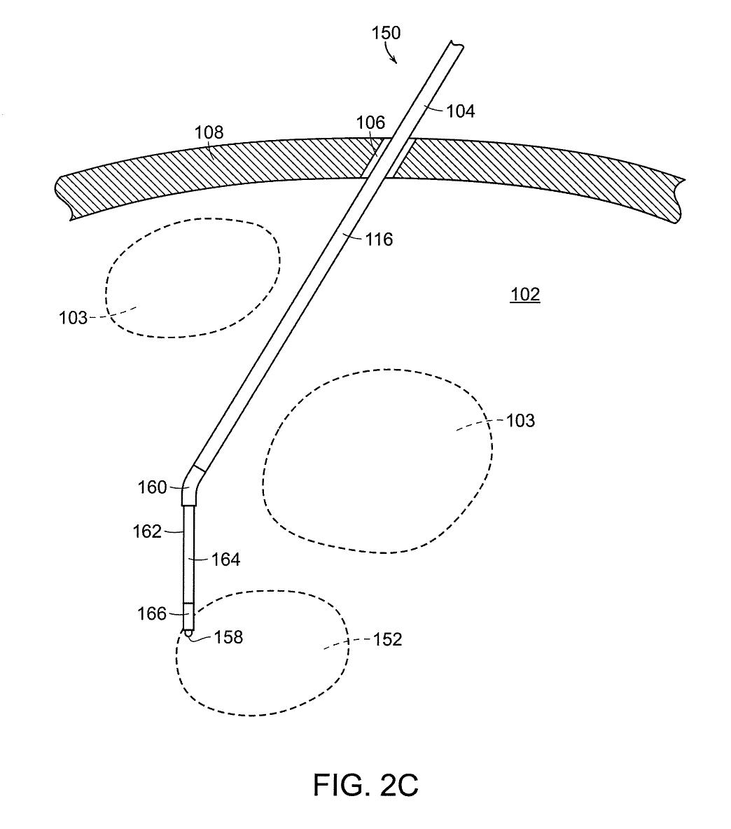

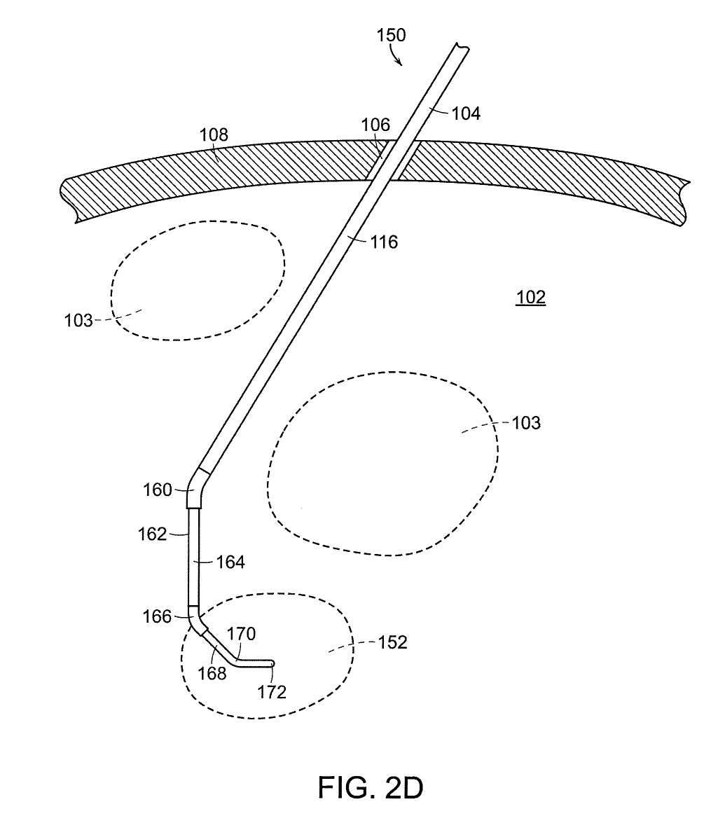

FIGS. 2A-2D illustrate a catheterization system 150 configured in accordance with another embodiment of the present technology. The catheterization system 150 of FIGS. 2A-2D is more highly articulated than the catheterization system 100 of FIGS. 1A-1C and is shown deployed into brain tissue 102 along a nonlinear path to a target area 152 having a different position than the target area 112 shown in FIGS. 1A-1C. The nonlinear path includes a first substantially straight portion 154 and a second substantially straight portion 156 with an angle between the first substantially straight portion and the second substantially straight portion. An obturator 158 that is slightly narrower and significantly more flexible than the obturator 114 shown in FIG. 1A is used to facilitate advancement of the cannula 104 along the first substantially straight portion 154 of the nonlinear path.

As shown in FIG. 2B, when a portion of the nonlinear path is reached where a change of direction is desired, an angle-forming member 160 is actuated to form a compact angle. Unlike the angle-forming member 118 shown in FIGS. 1A-1C, the angle-forming member 160 is actuated using pull wires, such as according to a pull-wire steering mechanism known in the art for vascular catheterization. The angle-forming member 160 can alternatively be pre-tensioned and actuated according to a process similar to the process described above with respect to the angle-forming member 118 shown in FIGS. 1A-1C. Similarly, a pull-wire steering mechanism can be used to actuate the angle-forming member 118 shown in FIGS. 1A-1C. The obturator 158 is flexible enough to conform to the angle of the angle-forming member 160. As shown in FIG. 2C, a second cannula 162 and the obturator 158 are then advanced through the cannula 104 and extended along the second substantially straight portion 156 of the nonlinear path. Like the cannula 104, the second cannula 162 includes a straight portion 164 and an angle-forming member 166 at its distal end. Unlike the cannula 104, the straight portion 164 of the second cannula 162 is not substantially rigid. The straight portion 164 of the second cannula 162 is flexible enough to allow it to pass through the angle-forming member 160 of the first cannula 104.

When the second cannula 162 reaches the target area 152, the angle-forming member 166 of the second cannula 162 can be actuated to form another compact angle. For example, the angle-forming member 166 can be pre-tensioned or actuated using a pull-wire steering mechanism. As shown in FIG. 1D, the obturator 158 is then withdrawn. A catheter 168 is advanced through the cannula 104, through the second cannula 162, and into the target area 152. The catheter 168 exits the second cannula 162 at a defined angle of the angle-forming member 166 and includes a joint 170 to control the position of a distal portion 172 of the catheter. Unlike the angle-forming members 160, 166, the joint 170 does not limit the catheter 168 to movement along a single path. The joint 170 is shown in FIG. 2D actuated to an angle of about 45°. The joint can have a range sufficient to allow the distal portion 172 of the catheter 168 to access all portions of the target area 152. For example, the joint 170 can have a range between about 120° and about 180°. In FIG. 2D, the joint 170 can have a range sufficient to allow the distal portion 172 of the catheter 168 to access portions of the target area 152 immediately adjacent to the angle-forming member 166.

The catheter 168 is more flexible than the angle-forming member 166. As discussed above, the straight portion 164 of the second cannula 162 is flexible enough to allow it to pass though the angle-forming member 160 of the first cannula 104. The angle-forming member 166 also is flexible enough to pass though the angle-forming member 160 of the first cannula 104 when the angle-forming member 166 is not actuated. Actuating the angle-forming member 166 can cause it to become more rigid. In combination, the actuated angle-forming member 166 and the straight portion 164 of the second cannula 162 can be rigid enough to maintain their position within the brain tissue 102 while the catheter 168 moves within the target area 152.

Several embodiments of the present technology include variations of the catheterization systems 100, 150 shown in FIGS. 1A-1C and 2A-D. For example, several embodiments include a greater number of cannulas to form paths having more than one angle. Additional cannulas can be deployed, for example, in a similar manner to the second cannula 162 shown in FIGS. 2C-2D. With a greater number of articulated or telescoping elements, devices and systems configured in accordance with several embodiments of the present technology can traverse virtually any path through CNS tissue in a constrained manner. Portions of the cannulas configured in accordance with several embodiments of the present technology can include radiopaque markers to facilitate navigation. For example, a straight portion or an angle-forming member of a cannula can include an elongated, radiopaque marker extending along a portion of the length of the straight portion or the angle-forming member. In several embodiments of the present technology, ring-shaped or partial-ring-shaped radiopaque markers are positioned at openings or at one or both ends of an angle-forming member.

The interaction between multiple cannulas can be different than the interaction between the cannula 104 and the second cannula 162 shown in FIGS. 2C-2D. For example, a second cannula can be positioned outside a first cannula and advanced along a second substantially straight portion of a catheterization path using a slightly wider obturator than the obturator 158 shown in FIGS. 2A-2C. Instead of a flexible obturator, an obturator used in several embodiments of the present technology can include a head that detaches from a substantially rigid body. A flexible member can extend through the substantially rigid body to push the head through an angle and along a path of a cannula. Such an obturator can be used, for example, with the catheterization system 150 shown in FIGS. 2A-2D. The head can be remotely detached when the end of the first substantially straight portion 154 of the nonlinear path is reached. Then head can then travel with the second cannula 162 along the second substantially straight portion 156 of the nonlinear path until the target area 152 is reached. The head can then be remotely withdrawn via the flexible member connecting the head to a remaining portion of the obturator.

Several embodiments of the present technology can include cannulas, catheters, and other elements having a variety of compositions and sizes. Suitable materials for substantially rigid elements, such as the straight portion 116 of the cannula 104 shown in FIGS. 1A-1C, include stainless steel and hard polymers. The composition of the second cannula 162 shown in FIGS. 2C-2D can include a reinforcing structure, such as a braided material (e.g., a braided metal wire) encased in a polymer, to allow flexibility and provide resistance to collapse. Smaller diameters are preferable for elements of several embodiments of the present technology, as they cause less disturbance of CNS tissue along the catheterization path. Cannulas or catheters of devices and systems configured in accordance with several embodiments of the present technology can have sizes between about 3 French and about 20 French, such as between about 5 French and about 14 French.

### 2. Catheterization Portal

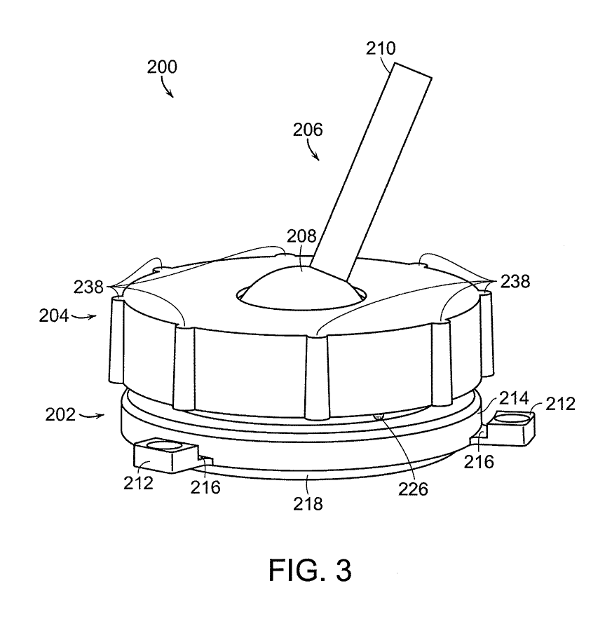

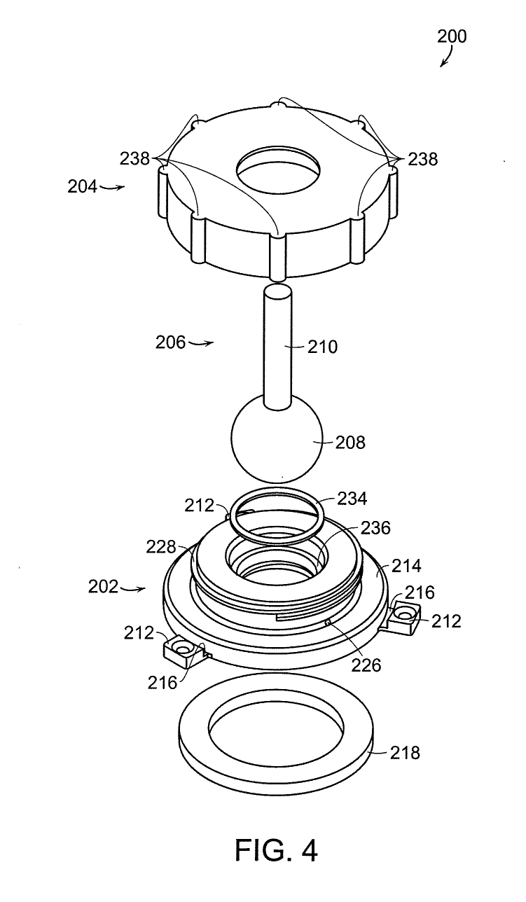

Devices and systems configured in accordance with several embodiments of the present technology can include a catheterization portal, such as a skull mount configured to provide rapid, precise, safe, and minimally invasive transcranial access. FIGS. 3-8 illustrate a skull mount 200 and portions thereof configured in accordance with an embodiment of the present technology. The skull mount 200 includes a base 202, a cap 204, and an adjustable portal 206. As shown in FIGS. 3 and 4, the adjustable portal 206 includes a spherical portion 208 and a directional portion 210. The spherical portion 208 is captured between the base 202 and the cap 204 to lock the adjustable portal 206 in a particular position. Similar to a ball-and-socket joint, prior to locking the spherical portion 208 between the base 202 and the cap 204, the position of the spherical portion can be adjusted to angle and radially position the directional portion 210. The maximum angle is the angle at which the cap 204 blocks further angling of the directional portion 210. In the skull mount 200, the maximum angle is about 30°. Alternative catheterization portals configured in accordance with several embodiments of the present technology can have greater or smaller radial ranges of motion between an adjustable portal and a fixed portion.

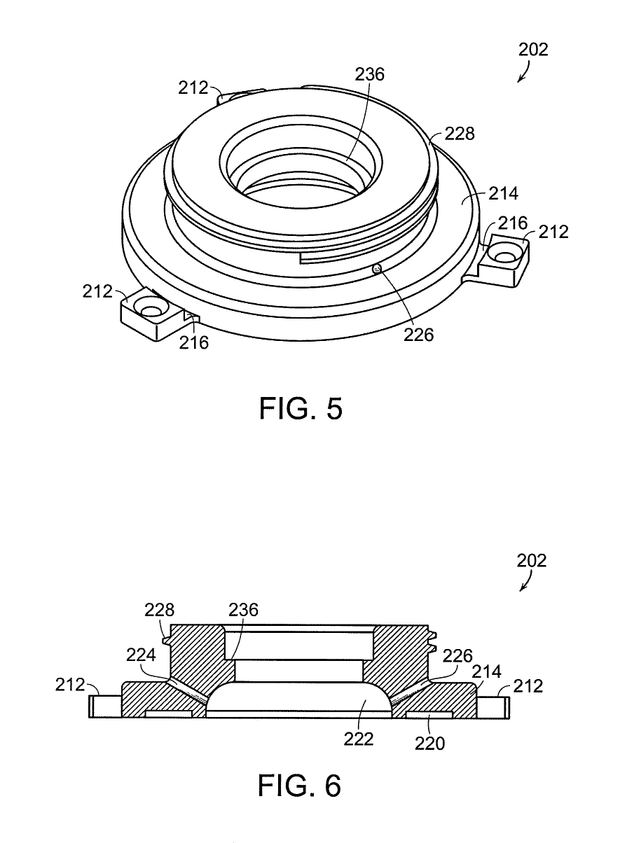

The skull mount 200 allows for the execution of a neurosurgical plan having a particular angle of entry into the brain. Furthermore, the skull mount 200 can be positioned at any portion of the scalp according to the specifications of a neurosurgical plan. As shown in FIGS. 5 and 6, the base 202 includes three mounting tabs 212 connected to a body 214 of the base with living hinges 216. The living hinges 216 can be made of a flexible plastic (e.g., polyethylene or polypropylene). In the illustrated skull mount 200, the mounting tabs 212 are sized to accommodate 3-millimeter diameter bone screws. The living hinges 216 help the base 202 conform to irregularities of a scalp surface. The skull mount 200 also includes a gasket 218 positioned within a gasket recess 220 on a bottom surface of the body 214 of the base 202. The gasket 218 can be sufficiently conformable to form a water-tight seal between the base 202 and an irregular surface of a scalp.

As shown in FIG. 6, the body 214 of the base 202 includes a chamber 222 configured to be positioned between a scalp surface and the spherical portion 208 of the adjustable portal 206. The body 214 includes an inlet conduit 224 and an outlet conduit 226. In operation, an inlet pipe (not shown) and an outlet pipe (not shown) can be connected to the inlet conduit 224 and the outlet conduit 226, respectively. The inlet and outlet pipes can be configured to create a continuous or intermittent flush of the chamber 222. For example, a flushing liquid (e.g., saline) can be introduced through the inlet conduit 224 and removed through the outlet conduit 226. Such flushing can help to clean the skull opening and prevent infection. Valves can be included to control the flow of a flushing fluid or to otherwise seal or unseal the chamber 222 as necessary. As another feature to minimize the risk of infection, the skull mount 200 can be disposable. For example, the skull mount 200 can be made primarily of a low-cost, hard plastic. If not disposable, portions of the skull mount 200 can be configured for thorough sterilization, such as in an autoclave.

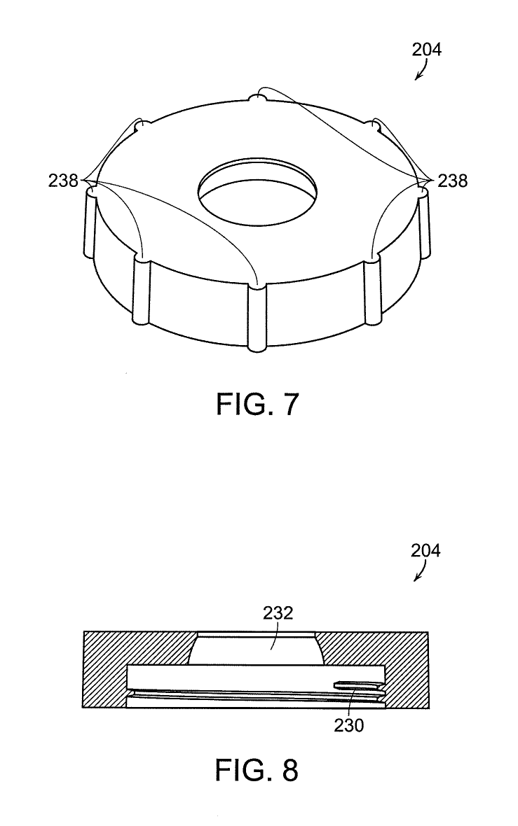

FIGS. 7 and 8 illustrate the cap 204 of the skull mount 200. To lock the cap 204 to the base 202, the cap can be rotated such that a male treaded portion 228 of the base interlocks with a female threaded portion 230 of the cap. The threads of the male treaded portion 228 and the female threaded portion 230 are of a trapezoidal, Acme profile. When the adjustable portal 206 is positioned within the skull mount 200, pressure from screwing the cap 204 onto the base 202 can press a clamping surface 232 of the cap against the spherical portion 208 of the adjustable portal, which can press the spherical portion into an o-ring 234 positioned on a seat 236 of the base. As shown in FIG. 8, the clamping surface 232 is concave with a curvature matching the curvature of the spherical portion 208 of the adjustable portal 206. Friction between the spherical portion 208 and the o-ring 234, between the o-ring and the seat 236, and between the spherical portion 208 and the clamping surface 232 can serve to lock the adjustable portal 206 in a particular position within the skull mount 200. As shown in FIG. 7, the cap 204 includes ridges 238 to aid in gripping the cap when locking the cap to the base 202.

Use of the skull mount 200 configured in accordance with several embodiments of the present technology can include placing the base 202 and the gasket 218 on a scalp of a patient at a selected site and inserting screws into screw holes of the mounting tabs 212. The adjustable portal 206 and the cap 204 then can be secured to the base 202 with the directional portion 210 pointed in a direction of a first portion of a planned catheterization path. A drill having a drilling member slightly larger or substantially similar in diameter to a cannula or catheter to be introduced into the brain then can be used to drill an opening in the skull. After drilling, the adjustable portal 206 and the cap 204 can be removed so that the site of the opening can be thoroughly cleaned of bone fragments. Alternatively, the flushing mechanism discussed above can be used to clean the site. A hand tool can be used to separate the dura matter or crush any hardened dura matter under the opening. Systems configured in accordance with several embodiments of the present technology can include such a hand tool as well as a drill or drill bit configured to form an opening having an appropriate diameter for insertion of a cannula or catheter of the system.

If the adjustable portal 206 and the cap 204 are removed for preparation of the skull opening, the position of the adjustable portal relative to the cap can be recreated. Alternatively, the adjustable portal 206 and the cap 204 can be fixed relative to each other (e.g., with epoxy glue) prior to their removal from the base 202 and then resecured to the base in the fixed configuration after preparation of the skull opening. Once the skull opening has been prepared, a cannula or catheter can be introduced into the brain via the adjustable portal 206. In catheterization portals configured in accordance with several embodiments of the present technology, one adjustable portal (e.g., the adjustable portal 206) is included for drilling and a second adjustable portal is included for catheterization. The second adjustable portal can include features to facilitate catheterization, such as a Tuohy-Borst adapter to prevent backflow. The second adjustable portal also can be configured to prevent unintentional movement of the catheter. For example, the second adjustable portal can features that frictionally engage the catheter and increase the threshold of force required to move the catheter in any direction (e.g., axially, laterally, or radially).

Catheterization portals configured in accordance with several embodiments of the present technology can be configured to allow an operator to manipulate a cannula or catheter while the operator is positioned at a significant distance from a patient's head. This can be useful to minimize the operator's exposure to radiation from data-gathering systems (e.g., fluoroscopy systems) in use during a procedure. In several embodiments of the present technology, an operator can manipulate a cannula or catheter when positioned between about 0.5 meter and about 5 meters from a patient's head, such as between about 1 meter and about 3 meters from a patient's head. In a neurosurgical procedure, preventing unintentional movement of a cannula or catheter within CNS tissue can be important to prevent damaging tissue around a catheterization path. Interaction between an elongated, rigid portal (e.g., the directional portion 210 of the adjustable portal 206) and a portion of a cannula or catheter extending into the CNS tissue can be useful in preventing such unintentional movement. For example, a rigid or flexible portion a cannula or catheter can fit snugly within the directional portion 210 of the skull mount 200 to prevent the cannula or catheter from moving in any direction other than forward or backward along the length of the directional portion. A directional portion of a skull mount configured in accordance with several embodiments of the present technology can have a length between about 5 times and about 100 times the diameter of a lumen within the directional portion, such as between about 10 times and about 50 times the diameter of the lumen.

Catheterization portals configured in accordance with several embodiments of the present technology can have a variety of features in addition to the features disclosed above and in FIGS. 3-8. For example, the catheterization portal can be substantially transparent to fluoroscopy or be substantially transparent to fluoroscopy except for one or more radiopaque markers to facilitate navigation. A radiopaque marker, for example, can be included to indicate a direction of an elongated portal (e.g., the directional portion 210 of the skull mount 200). Catheterization portals configured in accordance with several embodiments of the present technology also can include a portion of an ultrasonography system. As described in greater detail below, ultrasonography can be used to navigate a cannula or catheter within CNS tissue in accordance with several embodiments of the present technology. An ultrasound transducer or an array of ultrasound transducers can be positioned on the catheterization portal to monitor a cannula or catheter or to interact with a corresponding ultrasonography element on the cannula or catheter. For example, the catheterization portal can include an ultrasound transducer aligned with an elongated portal (e.g., the directional portion 210 of the skull mount 200). The ultrasound transducer can be positioned on a portion of the elongated portal or positioned on a separate structure adjustable to match the direction of the elongated portal. The catheterization portal also can include an ultrasound transducer that is manually or automatically adjustable to point toward a corresponding ultrasonography element on a portion of a cannula or catheter within CNS tissue. For example, an ultrasound transducer can be positioned on a mount having mechanical or magnetic actuators responsive to a manual or automatic control system. An automatic control system can include ultrasound data processing, such as proximity detection from A-mode ultrasound data. Ultrasound transducers on catheterization portals configured in accordance with several embodiments of the present technology can be configured for distance measurement or imaging. For example, catheterization portals configured in accordance with several embodiments of the present technology include ultrasound transducers configured for the collection of M-mode ultrasound data.

### 3. Catheter Features

Catheters configured in accordance with several embodiments of the present technology can have functional structures to treat target areas within the CNS. For example, the distal portions of such catheters can be configured to remove material while minimizing damage to surrounding tissue. This is particularly useful for removing clots occurring in healthy tissue. The surrounding tissue can be damaged, for example, by aggressive tearing or pulling of a clot. A clot targeted for removal is likely to be relatively large compared to the catheter. Cutting the clot into pieces outside the catheter can require aggressive mechanical action, which is likely to damage surrounding tissue. Clots often have significant surface integrity, so applying suction to an intact surface of a clot is likely to pull the clot excessively without necessarily breaking it into removable pieces. In contrast to these approaches, catheters configured in accordance with several embodiments of the present technology can be configured to carefully disrupt an object surface, such as by carving off portions of the object that are near a lumen of the catheter or protrude into the lumen of the catheter. Alternatively or in addition, the catheters can be configured to disrupt the object surface using another form of mechanical action (e.g., applied within or slightly outside a catheter lumen). Clot material, for example, can usually be drawn into a catheter through a disrupted surface with a minimal amount of suction.

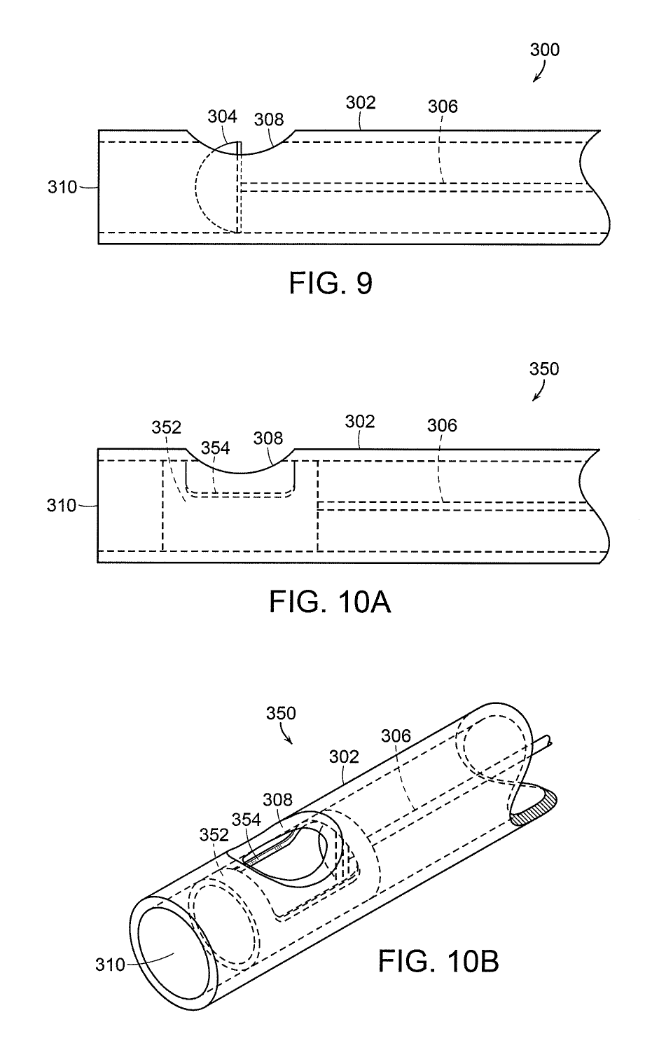

FIGS. 9-10B illustrate catheter distal portions configured in accordance with several embodiments of the present technology that are particularly well suited for carving off portions of an object (e.g., a clot). These embodiments, however, also can be used to disrupt object surfaces using another form of mechanical action among other functions. FIG. 9, for example, illustrates a catheter distal portion 300 including a body 302 at least partially defining a lumen. A surface disrupter 304 is positioned within the lumen at the distal end of a driver 306. The driver 306 is flexible and extends through the catheter to a manual or automatic actuator (not shown) beyond a proximal end of the catheter. The surface disrupter 304 in the illustrated embodiment has the form of a substantially rigid, cup-shaped cutter having a blunt, rounded distal end and a ring-shaped, cutting edge on its proximal side. The body 302 includes a lateral opening 308 and an end opening 310. Other embodiments can include no end opening as well as zero, two, three, or a greater number of lateral openings. The driver 306 is configured to move the surface disrupter 304 relative to the lateral opening 308 and the end opening 310 or to rotate the surface disrupter.

In operation, the catheter distal portion 300 can be positioned such that an object targeted for removal (e.g., a clot) is near the lateral opening 308 or the end opening 310. Suction can be applied to partially draw the object into the lateral opening 308 or the end opening 310. The driver 306 can move the surface disrupter 304 along the length of the catheter distal portion 300 or rotate the surface disrupter 304 to carve off a portion of the object or otherwise disrupt a surface of the object. The contact can occur within the lumen (e.g., if suction is used to draw the surface of the object through the lateral opening 308 or the end opening 310) or outside the lumen, such as slightly beyond the distal end. The driver 306 can press the surface disrupter 304 into the object or slightly rotate the surface disrupter 304 to disrupt the surface of the object. After the surface of the object has been disrupted, the driver 306 can withdraw the surface disrupter 304. Suction can then be applied to draw material from the object into the lumen through the object's disrupted surface and the lateral opening 308 or the end opening 310. Once material from an object targeted for removal is within the lumen, the material typically can be macerated or moved relatively aggressively without damaging surrounding tissue. For example, the surface disrupter 304 can be used to push or pull material within the lumen. The surface disrupter 304 also can be rotated at a relatively high speed while suction draws the material through the catheter. Macerating the material in this manner can be useful to facilitate movement of the material through the remaining length of the catheter without using strong suction.

In the catheter distal portion 300 shown in FIG. 9, the surface disrupter 304 has a distal rounded portion and a proximal straight portion. In other embodiments, the surface disrupter 304 can be reversed, with a proximal rounded portion and a distal straight portion. The surface disrupter 304 also can be replaced with another type of surface disrupter, such as a bullet-shaped surface disrupter having a distal tip. FIGS. 10A-10B illustrate another embodiment of a catheter distal portion 350 including a surface disrupter 352 in the form of a partial tube. The surface disrupter 352 includes a cutting surface 354 that is shaped or otherwise configured to generally correspond to the lateral opening 308. When suction is applied to draw a portion of an object (e.g., a clot) into the lumen, the driver 306 can move the surface disrupter 352 to carve off material from the object. The distal end of the surface disrupter 352 is open. In an alternative embodiment, the distal end of the surface disrupter 352 can be closed so as to capture material (e.g., a biopsy) within the surface disrupter. In the alternative embodiment, the surface disrupter 352 can be withdrawn after the material is captured and cleaned prior to reinsertion. The surface disrupters 304, 352 shown in FIGS. 9-10B occupy substantially the entire internal diameters of the lumens of the catheter distal portions 300, 350. Alternatively, the surface disrupters 304, 352 can be smaller than the internal diameters of the lumens of the catheter distal portions 300, 350.

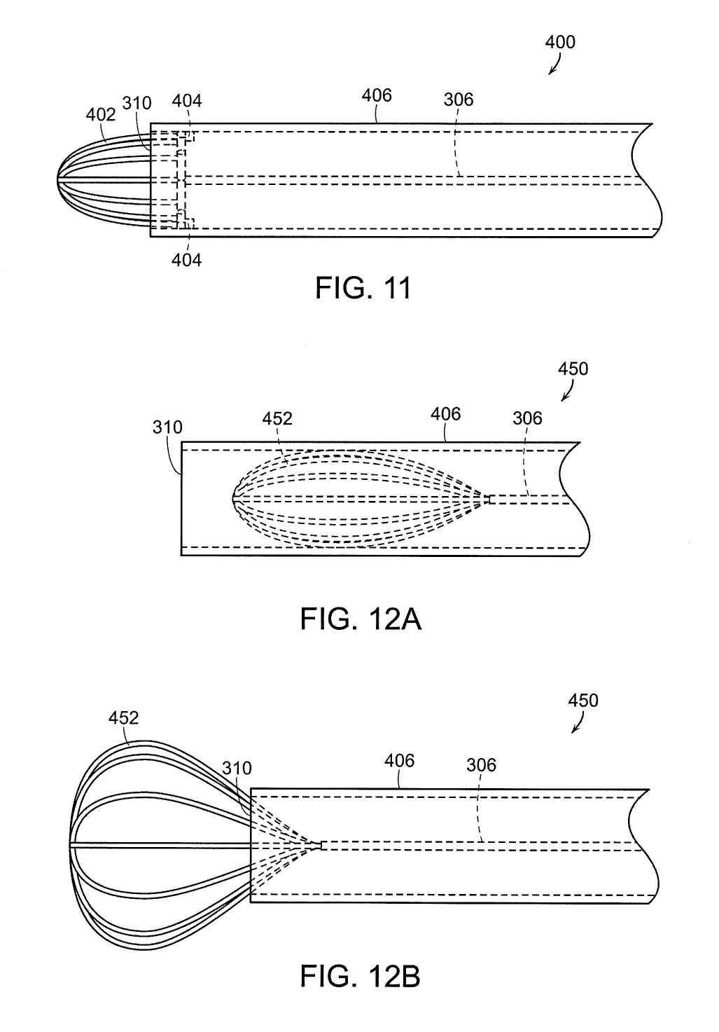

FIGS. 11-12B illustrate catheter distal portions configured in accordance with several embodiments of the present technology that are particularly well suited for disrupting object surfaces (e.g., clot surfaces) using gentle mechanical action. These embodiments, however, also can be used to carve off portions of an object among other functions. FIG. 11, for example, illustrates a catheter distal portion 400 including a surface disrupter 402 with multiple, arching wires. The surface disrupter 402 can resemble an egg beater or a whisk. Stops 404 are included within the lumen near the distal end to prevent the surface disrupter 402 from withdrawing into the lumen. The body 406 of the catheter distal portion 400 does not include a lateral opening. In several alternative embodiments, the surface disrupter 402 can be rigidly fixed to the distal end of the catheter distal portion 400 or free to withdraw fully into the catheter distal portion. In several embodiments, the surface disrupter 402 can serve at least in part to protect tissue from the direct application of suction or from an edge of the catheter distal portion 400. In these and other embodiments, the driver 306 can be omitted and motion of the catheter distal portion 400 can be used to drive the surface disrupter 402 into an object.

In most neurosurgical applications, it is desirable to advance a catheter of minimum diameter to minimize damage to tissue along the catheterization path. A structure larger than the diameter of the catheter, however, can be useful to execute a treatment at the target area. For example, treatment at the target area can involve the removal of an object (e.g., a clot) much larger than a distal portion of the catheter. An expanding structure can facilitate such treatments without enlarging the diameter of the catheter. FIGS. 12A-12B illustrate a catheter distal portion 450 including a surface disrupter 452 that expands after it exits the lumen. In FIG. 12A, the surface disrupter 452 is shown in a compact configuration prior to extension and expansion. In FIG. 12B, the surface disrupter 452 is shown in an expanded configuration subsequent to extension. The expanded configuration can be the relaxed shape of the surface disrupter 452. In alternative embodiments of the catheter distal portions 400, 450 shown in FIGS. 11-12B, a larger or smaller number of wires can be included in the surface disrupters 402, 452. The wires also can be replaced with other elongated structures, such as ribbon structures with sharp edges to achieve more aggressive disruption of object surfaces. In addition, flexible membranes can extend over the surface disrupters 402, 452 or portions thereof, such as to cause the surface disrupters to be more gentle.

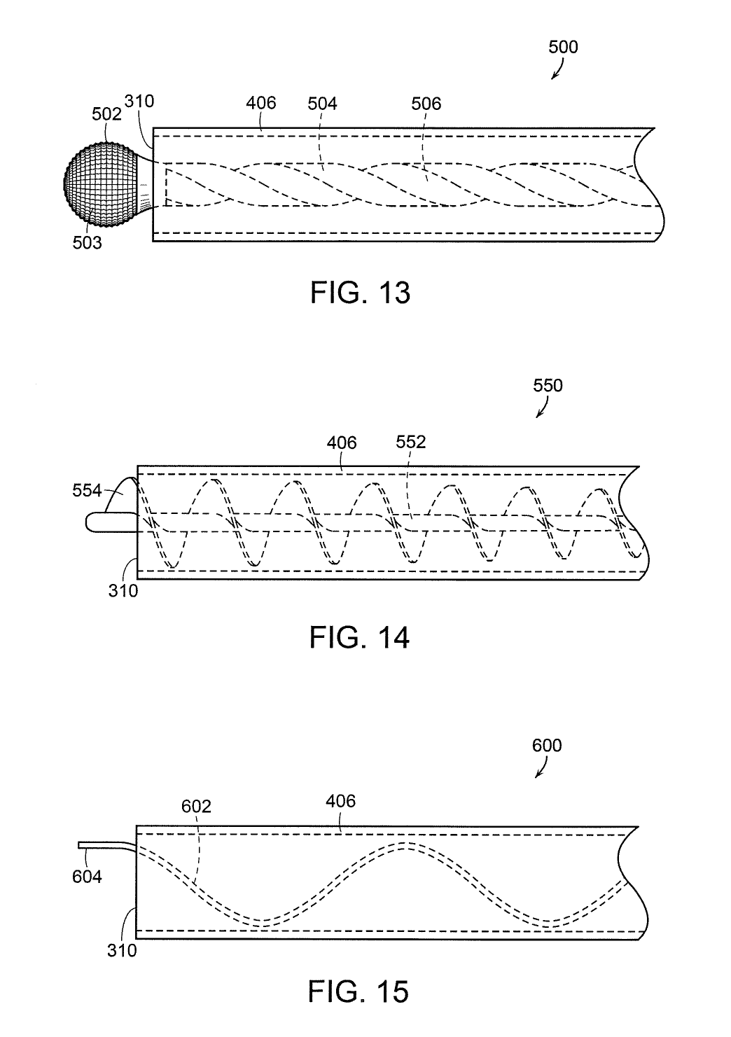

Catheter distal portions configured in accordance with several embodiments of the present technology can include structures that facilitate the removal material that enters the lumen. For example, such structures can be configured to macerate or move the material (e.g., as discussed above with reference to FIG. 9). FIGS. 13-15 illustrate catheter distal portions configured in accordance with several embodiments of the present technology that have such structures in combination with alternative structures well suited for disrupting object surfaces among other functions. FIG. 13 illustrates a catheter distal portion 500 including a surface disrupter 502 at the end of an elongated macerator 504 having a spiraling groove 506 similar to a twist drill bit. The surface disrupter 502 includes an abrasive pattern 503. The elongated macerator 504 has a larger diameter than the driver 306 shown in FIGS. 9-12B. The spiraling groove 506 can help to macerate material and act as a screw conveyor to move material through the lumen when the elongated macerator 504 is rotated. To be capable of advancing through angles along the catheterization path, the elongated macerator 504 can be flexible or can extend a short distance along the length of the catheter distal portion 500 prior to tapering or otherwise transitioning into a smaller-diameter driver similar to the driver 306 shown in FIGS. 9-12B. The elongated macerator 504 can transfer axial or rotational movement from a driver to the surface disrupter 502. The abrasive pattern 503 can have a degree of coarseness (e.g., a grit equivalent) corresponding to the requirements of a particular treatment application. A molding process, for example, can be used to form the abrasive pattern 503 to have having varying degrees of coarseness.

FIG. 14 illustrates a catheter distal portion 550 including an elongated macerator 552 including an Archimedean screw. An end portion 554 of the elongated macerator 552 extends slightly beyond the distal end of the catheter distal portion 550. In operation, the elongated macerator 552 can be moved along the length of the catheter distal portion 550 or rotated. The end portion 554 can disrupt the surface of an object near the distal end of the catheter distal portion 550 and therefore act as a surface disrupter. The Archimedean screw can help to macerate material and act as a screw conveyor to move material through the lumen when the elongated macerator 552 is rotated. The Archimedean screw tapers in diameter as it extends away from the end portion 554 and the elongated macerator 552 can eventually transition into a smaller diameter driver similar to the driver 306 shown in FIGS. 9-12B.

FIG. 15 illustrates a catheter distal portion 600 including an elongated macerator 602 in the form of a wire whip. An end portion 604 of the elongated macerator 602 extends slightly beyond the distal end of the catheter distal portion 600. In operation, the elongated macerator 602 can be moved along the length of the catheter distal portion 600 or rotated. The end portion 604 can disrupt the surface of an object near the distal end of the catheter distal portion 600 and therefore act as a surface disrupter. Rotation of the elongated macerator 602 within the lumen of the catheter distal portion 600 can help to macerate or move material to be transported through the catheter. Alternatively or in addition to rotating, the elongated macerator 602 can be configured to straighten partially or fully and then resiliently return to its spiraling shape. Pulling a proximal portion of the elongated macerator 602 can cause this action. The elongated macerator 602 is shown in FIG. 15 occupying almost the entire internal diameter of the lumen of the catheter distal portion 600. Alternatively, the elongated macerator 602 can occupy a smaller portion of the internal diameter of the lumen of the catheter distal portion 600.

The elongated macerator 602 can be flexible and extend along all of any portion of the length of the catheter, not just the catheter distal portion 600. The flexibility of the elongated macerator 602 can allow it to move through angles of the catheterization path. Several embodiments of the present technology include elongated macerators having more than one wire whip, such as two wire whips configured to rotate in opposite directions. Alternative embodiments can include elongated macerators having structures other than the wire whip shown in FIG. 15 that also remain flexible along the length of the catheter. Such elongated macerators can include, for example, a spiraling ribbon with or without sharpened edges. The macerating features (e.g., wire bends or sharpened edges) of such structures can be continuous or limited to one or more positions along the length of the catheter. For example, fewer macerating features may be useful near proximal portions of the catheter.

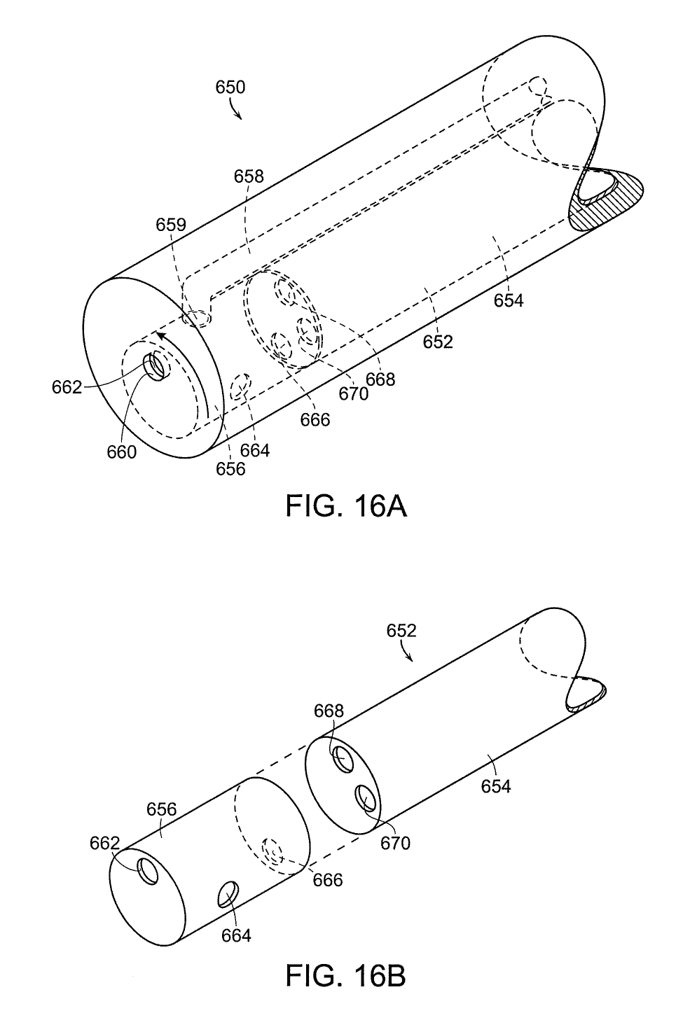

Catheter distal portions configured in accordance with several embodiments of the present technology can be designed to make use of suction, such as intermediately applied suction. The suction can be applied, for example, through the overall lumen of the catheter distal portion or through the lumen of a separate conduit within the lumen of the catheter distal portion. FIG. 16A illustrates a catheter distal portion 650 including a suction conduit 652 having a main portion 654 and a rotatable plug 656. The catheter distal portion 650 also includes a smaller flush conduit 658 having an end opening 659 abutting a lateral side of the rotatable plug 656. The main portion 654 of the suction conduit 652, the rotatable plug 656 of the suction conduit 652, and the flush conduit 658 can work together to apply suction in a highly controlled manner. The distal end of the catheter distal portion 650 includes a window 660. The rotatable plug 656 includes a distal window 662, a lateral window 664, and a proximal window 666. A distal end of the main portion 656 of the suction conduit 652 includes a first window 668 and a second window 670. FIG. 16B is an exploded perspective view of the suction conduit 652 showing the windows 662, 664, 666, 668, 670 with greater clarity than in FIG. 16A. A driver (not shown) similar to the driver 306 shown in FIGS. 9-12B can be connected to a proximal end of the rotatable plug 656 and extend proximally along the length of the suction conduit 652 for rotational actuation of the rotatable plug.

When the rotatable plug 656 is in a first position, as shown in FIGS. 16A-16B: (1) the window 660 of the catheter distal portion 650 and the distal window 662 of the rotatable plug are aligned, (2) the lateral window 664 of the rotatable plug and the end opening 659 of the flush conduit 658 are not aligned, and (3) the proximal window 666 of the rotatable plug is not aligned with either the first window 668 or the second window 670 of the main portion 654. In the this position, a controlled amount of suction corresponding to the vacuum pressure of a lumen of the rotatable plug 656 can be applied to draw material (e.g., clot material) into the lumen of the rotatable plug. The rotatable plug 656 then can be rotated 90° into a second position in which: (1) the window 660 of the catheter distal portion 650 and the distal window 662 of the rotatable plug are not aligned, (2) the lateral window 664 of the rotatable plug and the end opening 659 of the flush conduit 658 are aligned, and (3) the proximal window 666 of the rotatable plug and the second window 670 of the main portion 654 are aligned. In the this position, suction can be applied to the main portion 654 to draw material from the lumen of the rotatable plug 656, through the proximal window 666 of the rotatable plug, through the second window 670 of the main portion, into a lumen of the main portion, and along the length of the catheter. In addition, the suction can draw a flushing material (e.g., water) from the flush conduit 658, through the end opening 659 of the flush conduit, through the lateral window 664 of the rotatable plug 656, through the proximal window 666 of the rotatable plug, through the second window 670 of the main portion 654, into the lumen of the main portion, and along the length of the catheter. Once the lumen of the rotatable plug 656 has been flushed, the rotatable plug can be rotated 90° into a third position in which: (1) the window 660 of the catheter distal portion 650 and the distal window 662 of the rotatable plug are not aligned, (2) the lateral window 664 of the rotatable plug and the end opening 659 of the flush conduit 658 are not aligned, and (3) the proximal window 666 of the rotatable plug and the first window 668 of the main portion 654 are aligned. In this position, the lumen of the rotatable plug 656 can be charged with suction prior to repeating the process. Since the window 660 of the catheter distal portion 650 and the distal window 662 of the rotatable plug 656 are not aligned in the second and third positions, the suction used to flush the lumen of the rotatable plug and charge the lumen of the rotatable plug can be relatively strong.

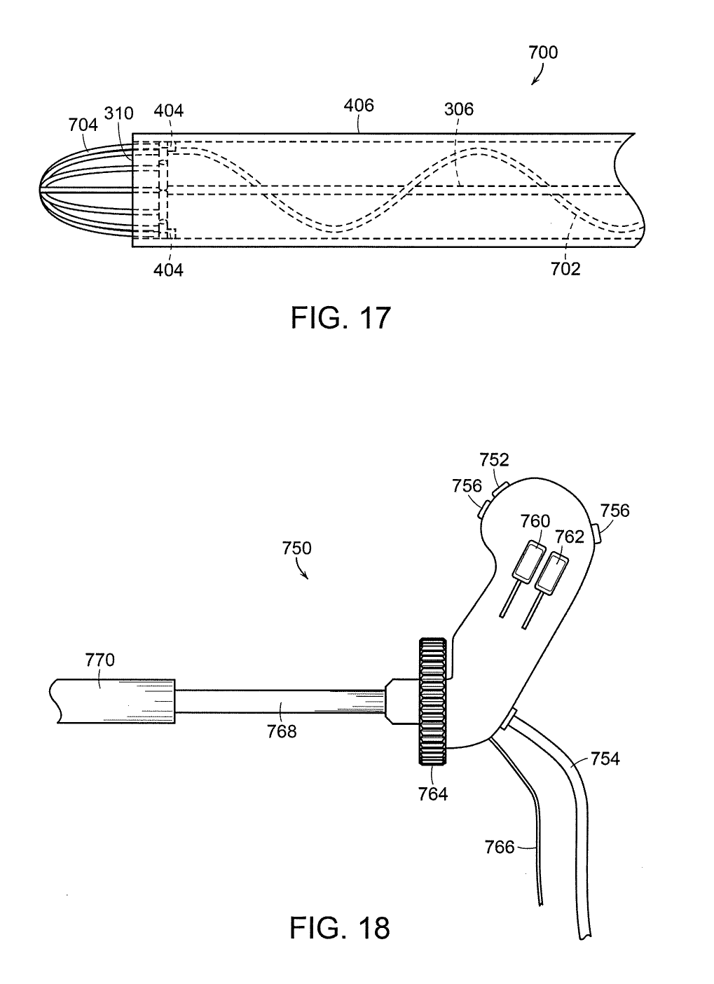

The various structures shown in FIGS. 9-16B can be removed, added, combined, or otherwise interchanged to create additional useful embodiments of catheters configured in accordance with the present technology. For example, various surface disrupters can be combined with various elongated macerators. FIG. 17 illustrates a catheter distal portion 700 including an elongated macerator 702 similar to the elongated macerator 602 shown in FIG. 15 and a surface disrupter 704 similar to the surface disrupter 402 shown in FIG. 11. In operation, the surface disrupter 704 can disrupt the surface of an object (e.g., a clot). Material from the object then can be drawn into the lumen of the catheter distal portion 700 and the elongated macerator 702 can macerate the material to facilitate its movement by suction through a remainder of the length of the catheter. The elongated macerator 702 also can be configured to extend slightly beyond the distal end of the catheter distal portion 700. For example, the elongated macerator 702 can be configured to extend to an area within the surface disrupter 704 and the surface disrupter can block further extension of the elongated macerator. Similarly, the surface disrupter 704 or a similar structure can be included in any of the catheter distal portions 300, 350, 450, 500, 550 shown in FIGS. 9-10B and 12A-14 to restrict movement of the surface disrupters 304, 352, 452, 502 and the end portion 554 beyond the distal ends of the catheter distal portions. For example, the surface disrupter 704 or a similar structure can be fixed to a distal end of the catheter distal portions 300, 350, 450, 500, 550 shown in FIGS. 9-10B and 12A-14.

In an example of a particularly advantageous combination in accordance with several embodiments of the present technology, the surface disrupter 704 or a similar structure is fixed to a distal end of the catheter distal portion 300 shown in FIG. 9. The surface disrupter 704 can restrict movement of the surface disrupter 304 and act as a screen through which material (e.g., clot material) can be drawn. The proximally facing sharpened edge of the surface disrupter 304 can cut material from an object extending through openings of the surface disrupter 704 (e.g., between wires of the surface disrupter 704) as the surface disrupter 304 is moved axially relative to the surface disrupter 704.

Several embodiments of the present technology include a catheter control assembly. This can include, for example, a hand controller having controls that facilitate tactile operation while an operator is concentrating on navigation or tissue-monitoring data. FIG. 18 illustrates a catheter controller 750 configured in accordance with an embodiment of the present technology. The catheter controller 750 includes a suction trigger 752 that can be used to activate suction through the catheter. An external suction source (not shown) can provide the suction through the suction conduit 754. Activating the suction can include automatically opening a valve between the suction conduit 754 and a lumen of the catheter when the suction trigger 752 is pressed. When the suction trigger 752 is released, the valve can automatically close. In this way, suction can be administered intermittently in discrete volumes. In operation, suction can be administered continuously to debulk an object (e.g., a clot) and then intermittently near edges of the object so that greater care can be taken to avoid disturbing surrounding tissue. Alternative embodiments can include multiple suction sources having different levels of suction. For example, the suction trigger 752 in the catheter controller 750 can be replaced with a strong-suction button configured to open a valve to a strong-suction conduit and a low-suction button configured to open a valve to a low-suction conduit. Such embodiments, for example, can allow the use of gentle suction for the removal of material and aggressive suction for flushing the catheter, as discussed above with reference to FIGS. 16A-16B.

The catheter controller 750 also includes an elongated macerator rotation trigger 756 and an elongated macerator sliding trigger 758. The elongated macerator rotation trigger 756 can be configured to rotate an elongated macerator in the catheter. The elongated macerator sliding trigger 758 can be configured to move the elongated macerator axially along the length of the catheter. Mechanical actuators within the catheter controller 750 can cause the rotation and movement in response to the elongated macerator rotation trigger 756 and the elongated macerator sliding trigger 758. Alternatively, a manual extension can allow manual control of rotation or axial movement of the elongated macerator. Other structures in catheters configured in accordance with several embodiments of the present technology, such as the surface disrupter 402 described above with reference to FIG. 11, also can be rotated or moved manually, such as with a crank. Rotation and movement of an elongated macerator can be used as needed to prevent occlusion of a lumen of the catheter. In alternative embodiments, the elongated macerator rotation trigger 756 and the elongated macerator sliding trigger 758 can be replaced or supplemented with other actuation triggers for other structures within the catheter. For example, the hand controller 750 can be used with a catheter having the suction conduit 652 described above with reference to FIGS. 16A-16B and the hand controller can include a trigger for rotating the rotatable plug 656, such as in 90° increments.

A first catheter joint control 760 and a second catheter joint control 762 on the catheter controller 750 each control an angle of a catheter joint, such as the joint 170 described above with reference to FIG. 2D. In other embodiments, no joint controllers, one joint controller, or more than two joint controllers can be included depending on the number of joints in the catheter. The first and second catheter joint controls 760, 762 include slides that can be positioned along a track to actuate different angles for the corresponding joints via pull wires. A rotation control 764 at the base of the catheter controller 750 can control rotation of the catheter. Such rotation can occur manually or via mechanical actuators within the catheter controller 750. A power conduit 766 supplies power for all structures of the catheter and catheter controller 750 that require power. In several embodiments having catheter elements that require power or generate signals (e.g. ultrasound signals), one or more electrical conduits for power delivery to or signal transmission from elements of the catheter can extend along the length of the catheter. For simplicity, such conduits are not shown in the Figures.

FIG. 18 illustrates a shaft 768 extending from the catheter controller 750 into an extension sleeve 770. The shaft 768 and the extension sleeve 770 are substantially rigid. In the illustrated embodiment, the shaft 768 is connected to a flexible portion of the catheter. The extension sleeve can be fixed during a neurosurgical procedure, such as to a floor mount or to a firm table mount. A distal end of the extension sleeve can be connected to a skull mount, such as the skull mount 200 described above with reference to FIGS. 3-8. Advancing and withdrawing the shaft 768 relative to the extension sleeve 770 can advance or withdraw the catheter within the CNS tissue.

Catheters, including catheter distal portions, configured in accordance with several embodiments of the present technology can have a variety of features in addition to the features disclosed above and in FIGS. 9-18. For example the catheters can include zero, one, two, three, or a greater number of joints to provide varying levels of maneuverability. Portions of the catheters can include radiopaque markers to facilitate navigation. Catheters configured in accordance with several embodiments of the present technology include cooling, heating, or ablation (e.g., ultrasound, radiofrequency, or microwave ablation) structures, such as at the tip of the catheters. A cooling structure, for example, can include a thermoelectric cooler or a conduit for recirculating coolant from an external refrigeration unit. Although illustrated primarily with straight-cut distal ends, catheter distal portions configured in accordance with several embodiments of the present technology can have distal ends having a variety of shapes, such as rounded, pointed, or angled.

Catheters configured in accordance with several embodiments of the present technology can include internal conduits for aspiration or delivery. For example, FIGS. 16A-16B illustrate a suction conduit 652 and a flush conduit 658. In other embodiments, a delivery conduit can be included for the delivery of a contrast agent (e.g., an intravascular contrast agent) or a drug (e.g., a hemostatic agent). Removal of a clot can reinitiate bleeding. To treat this bleeding and other forms of bleeding, fibrin glue can be delivered in two parts, with each part delivered through a separate conduit. The two parts can be mixed near the distal end of the catheter. A delivery conduit also can be included to deliver a liquid (e.g., saline) to the CNS tissue to maintain a pressure equilibrium. For example, suction of material can cause a negative pressure within a portion of the CNS, such as the skull cavity. If air is drawn in through the catheterization portal, it can negatively affect ultrasonography. A biologically inert liquid, however, such as saline can compensate for the pressure lost to suction without affecting ultrasonography. A slight positive pressure on the liquid can ensure that the liquid rather than air will offset any negative pressure in the CNS tissue. Other than for maintaining a pressure equilibrium, a liquid flush can be useful as part of a treatment. A liquid flush also can be used to remove material from the catheter. For example, a catheter opening can be blocked and a liquid introduced into a portion of the catheter, such as a distal portion of the catheter, to force material out of the catheter. Aspiration or delivery conduits can be within catheters configured in accordance with several embodiments of the present technology or used in place of such catheters. For example, aspiration or delivery conduits can be introduced through a cannula after a catheter is removed.

### 4. Navigation and Monitoring

Data acquisition including fluoroscopy or ultrasonography can be used to navigate the cannula or catheter along a catheterization path as well as to monitor surrounding tissue. Several embodiments of the present technology include data acquisition that accounts for shifts of the brain and surrounding structures in real time. Other data acquisition can be real time or delayed. Fluoroscopy used in several embodiments of the present technology can include any type of fluoroscopy known in the art, including CT fluoroscopy, flat-panel CT fluoroscopy, and 3D-biplane fluoroscopy. Catheters configured in accordance with several embodiments of the present technology can be configured to deliver contrast (e.g. intravascular contrast) via a delivery conduit to aid imaging. The combination of fluoroscopy and ultrasonography can be especially effective. For example, fluoroscopy can be used for primary navigation and ultrasonography (e.g., A-mode ultrasonography) can be used for confirmation or small-scale imaging. An ultrasonography system including an ultrasonography element mounted on the tip of a catheter can provide precise edge detection (e.g. sub-millimeter edge detection of an interface between brain tissue and clot material) during a procedure to supplement large-scale imaging (e.g., fluoroscopy).

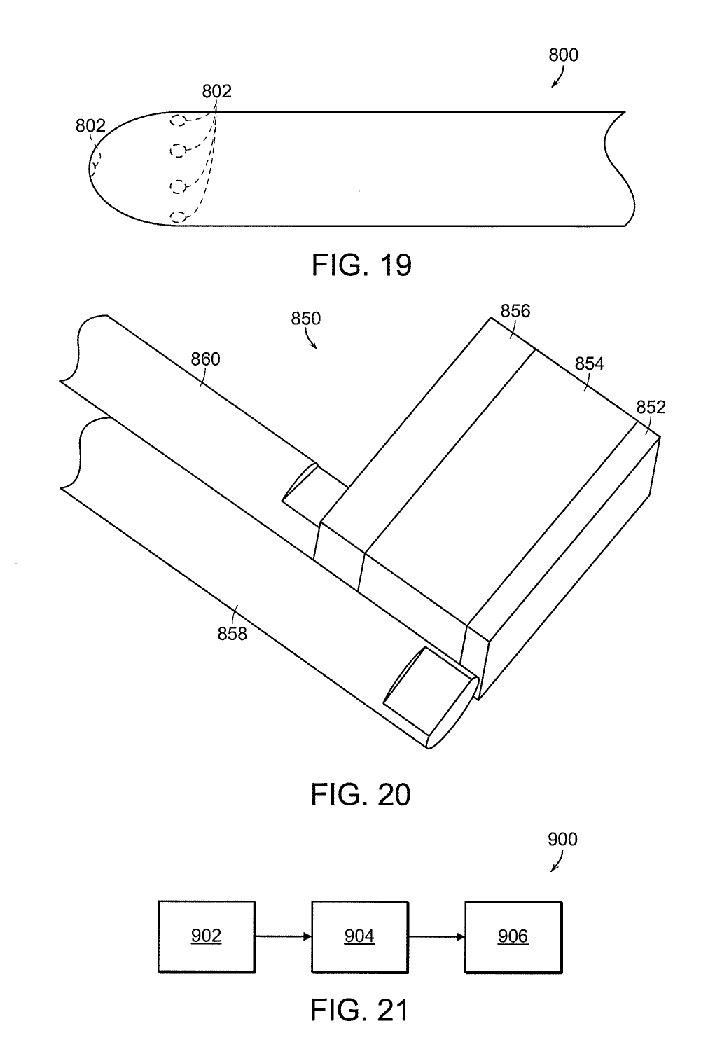

Devices and systems configured in accordance with several embodiments of the present technology can include one or more ultrasound transducers on an element intended to advance through CNS tissue, such as a cannula or catheter. FIG. 19, for example, illustrates a catheter distal portion 800 having a tip ultrasound transducer 802 and a series of radial ultrasound transducers 804. The tip ultrasound transducer 802 and the radial ultrasound transducers 804 can be configured for A-mode ultrasonography or another ultrasound modality. When an emitter and a receiver are the same ultrasound transducer or are located in close proximity, A-mode ultrasonography or another ultrasound modality can be used to determine a distance to a target (e.g., a clot) having a different acoustic impedance than adjacent tissue. A-mode ultrasonography can be particularly useful at least in part due to its simplicity and its compatibility with the miniaturized dimensions of catheters configured in accordance with several embodiments of the present technology. Although typically not suitable for complex imaging, A-mode data can be sufficient, for example, to confirm that a catheter is moving toward a target or to detect whether a catheter performing a mechanical thrombectomy has reached the edge of a clot. For example, data from the tip ultrasound transducer 802 and the radial ultrasound transducers 804 can be monitored in real time during a mechanical thrombectomy. If any of the tip ultrasound transducer 802 and the radial ultrasound transducers 804 indicate a distance to a brain-to-clot interface less than a threshold distance (e.g., 1, 2, 3, 4, or 5 millimeters), the procedure can be stopped or slowed as necessary before damage to tissue surrounding the clot can occur.

In several embodiments of the present technology, A-mode ultrasonography is used in conjunction with fluoroscopy. In fluoroscopy, clot material typically is not differentiated from brain tissue. Fluoroscopy also typically does not provide real-time data. Fluoroscopy images can be taken periodically during a procedure. At any point during a mechanical thrombectomy, the most recent fluoroscopy image stored for observation can cease to reflect accurately the location of a brain-to-clot interface. Ultrasound data indicating that a brain-to-clot interface is no longer where it is expected to be can prompt the neurosurgeon to refresh the fluoroscopy image. In addition, the resolution of a fluoroscopy image, which often is displayed on a monitor at some distance from the neurosurgeon, typically is significantly lower than the resolution of A-mode ultrasonography. In accordance with several embodiments of the present technology, a neurosurgeon can move a catheter close to a target using fluoroscopy and then use ultrasonography to achieve higher resolution guidance. Ultrasonography also can compensate for the lack of depth perspective in a 2-D fluoroscopy image. When a neurosurgeon is looking at a 2-D fluoroscopy image, the catheter can be in a different plane than the image. As the catheter is apparently moved toward a target, the catheter can actually be in front of or behind the target and can be encroaching on a brain-to-clot interface. Ultrasound data (e.g., A-mode ultrasound data) can provide confirmation that a brain-to-clot interface is at an expected location or warning that a brain-to-clot interface is not at an expected location. Such a warning can prompt the neurosurgeon to obtain a fluoroscopy image from a different plane.

FIG. 20 illustrates a specific example of an ultrasound transducer assembly suitable for use in the tip of a catheter distal portion configured in accordance with several embodiments of the present technology. The illustrated ultrasound transducer assembly 850 includes a transducer structure 851 having a front layer 852, a center layer 854, and a back layer 856. A ground lead 858 and a positive lead 860 are connected to the front layer 852 and the back layer 856, respectively. The front layer 852 is a quarter-wave acoustic matching layer having a thickness of 0.048 millimeter. The center layer 854 is a Pz27 ceramic piezoelectric layer having a thickness of 0.215 millimeter. The back layer 856 has a thickness of 0.096 millimeter. The ground lead 858 and the positive lead 860 are 36 AWG multifilar magnet wires having a diameter of 0.1397 millimeter. Electrical connections (not shown) extend between the tips of the ground lead 858 and the positive lead 860 and the front layer 852 and the back layer 856, respectively. The electrical connections, the front layer 852, and the back layer 856 are made of conductive epoxy. An epoxy encapsulant (not shown) surrounds the transducer structure 851 and the electrical connections. The transducer structure 851 is designed to operate at a center frequency of 10 MHz. The face dimensions of the transducer structure 851 are 0.5 millimeter by 0.25 millimeter. In a test using a glass plate as a reflection boundary, the ultrasound transducer assembly 850 was found to have a position resolution of about 0.010 millimeter. Catheters in accordance with several embodiments of the present technology can include an ultrasound transducer having a center frequency between about 5 MHz and about 20 MHz, such as between about 7 MHz and about 15 MHz or between about 8 MHz and about 12 MHz. The center frequency can be selected, for example, to provide the optimal differentiation of clot material relative to brain tissue with the minimum amount of noise, e.g., from bubbles.

Ultrasonography systems configured in accordance with several embodiments of the present technology can include components positioned externally during a procedure. For example, instead of a single ultrasound transducer in a catheter acting as an emitter and a receiver, an ultrasound transducer acting as an emitter can be positioned in a catheter and an ultrasound transducer acting as receiver can be positioned externally, such as on a skull mount. Alternatively, an ultrasound transducer acting as a receiver can be positioned in a catheter and an ultrasound transducer acting as a receiver can be positioned externally, such as in a skull mount. When an emitter and a receiver have different locations, A-mode ultrasonography or another ultrasound modality can be used to determine a distance between the emitter and the receiver. Skull mounts configured in accordance with several embodiments of the present technology can include mechanical actuators configured to move an ultrasonography element to track the position of a corresponding ultrasonography element on a catheter deployed in CNS tissue. Ultrasonography systems configured in accordance with several embodiments of the present technology including an element on the catheter and a fixed external element can provide the operator with an accurate three-dimensional report of the direction the portion of the catheter is moving, such as the direction a tip of the catheter is bending.

Several embodiments of the present technology can include elements configured for shear-wave ultrasound imaging, such as to detect or refine detection of a brain-to-clot interface. Shear-wave ultrasound imaging can include depositing enough ultrasound energy to stimulate in the CNS tissue a shear wave that propagates at a velocity two to three orders of magnitude slower than the longitudinal waves. An ultrasound transducer on a skull mount can provide the ultrasound energy. A rapid succession of longitudinal wave pulses can be used to monitor propagation of the shear wave. In this way, shear-wave-induced tissue displacements can be detected and correlated to the elastic modulus of portions of the CNS and surrounding structures to generate useful data for navigation or monitoring. Such data can be used, for example, to detect or measure the volume of a target object (e.g., a clot), to detect or measure the stiffness of a target object, to detect the position of a catheter within a target object, or to identify a structure directly adjacent to a catheter (e.g. as clot or brain tissue).

In addition to or instead of fluoroscopy and ultrasonography, several embodiments of the present technology can include other forms of data acquisition. For example, data from diffusion tensor imaging can be used to plan and execute a catheterization path that minimizes damage to specific fiber tracks. Several embodiments of the present technology also can include elements for electromagnetic surgical guidance (e.g., STEALTH surgical guidance). For example, catheters configured in accordance with several embodiments of the present technology can include a wire-mounted antenna or a separate antenna in a distal portion of the catheter (e.g., the distal tip). Such an antenna can be located adjacent to an ultrasound transducer. Catheters in accordance with several embodiments of the present technology also can include an optical imaging component in place of or in addition to an ultrasound transducer. For example, the distal end of a catheter in accordance with several embodiments of the present technology can include a light source and a photodetector.

Data from fluoroscopy, ultrasonography, or other sources can be included on a display, such as a graphic user interface. The display can be real time or delayed. Several embodiments of the present technology include a display having a known dimensional scale, such as a dimensional scale set by the operator for greater or less precision. A display in several embodiments of the present technology also can include a representation of intracranial anatomy. When available, ultrasound data can be combined with fluoroscopy data on a single display. Alternatively, ultrasound and fluoroscopy data can be displayed separately. FIG. 21 illustrates an ultrasonography system configured in accordance with several embodiments of the present technology. The ultrasonography system 900 includes a source of ultrasound data 902 (e.g., an ultrasound transducer in a catheter or an ultrasound transducer on a skull mount), a processing system 904, and a display 906. The processing system 904 can be configured to receive the ultrasound data and to translate it into a suitable form for display. For example, amplitude data can be converted into distance measurements.

From the foregoing, it will be appreciated that specific embodiments of the present technology have been described herein for purposes of illustration, but that various modifications can be made without deviating from the spirit and scope of the disclosure. For example, the catheterization system 100 shown in FIGS. 1A-1C and the catheterization system 150 shown in FIGS. 2A-2D each can be used with a catheter including any of the catheter distal portions 300, 350, 400, 450, 500, 550, 600, 650, 700, 800 shown in FIGS. 9-17 and 19. Aspects of the disclosure described in the context of particular embodiments can be combined or eliminated in other embodiments. For example, the flush conduit 658 can be eliminated from the catheter distal portion 650 shown in FIGS. 16A-16B and the various windows can be modified such that the rotatable plug 656 of the suction conduit 652 transitions between only two positions: a suction-charging position and a suction-application position. With this modification, the entire catheter distal portion 650 can serve as a suction conduit and the remaining windows can be enlarged. Further, while advantages associated with certain embodiments of the disclosure have been described in the context of those embodiments, other embodiments may also exhibit such advantages, and not all embodiments need necessarily exhibit such advantages to fall within the scope of the disclosure. Accordingly, embodiments of the disclosure are not limited except as by the appended claims.

I/We claim:

1. A neurosurgical catheter, comprising:
a body having a lumen; and
a surface disrupter movable within the lumen along a length of the neurosurgical catheter and extendable from a distal end of the lumen, wherein the surface disrupter includes a distal portion and a proximal portion, wherein the distal portion is substantially blunt, and wherein the proximal portion includes a cutting edge.

2. The neurosurgical catheter of claim 1, wherein the surface disrupter at least partially defines a recess, and wherein the cutting edge is a ring around an opening of the recess.

3. The neurosurgical catheter of claim 1, wherein both the distal portion and the proximal portion of the surface disrupter are extendable beyond the distal end of the lumen.

4. The neurosurgical catheter of claim 1, further comprising a lateral opening extending through a wall of the body and into the lumen at a distal portion of the neurosurgical catheter, wherein the surface disrupter is movable within the lumen such that at least a portion of the surface disrupter slides through the lumen proximate the lateral opening.

5. The neurosurgical catheter of claim 1, wherein the blunt distal end is substantially convex, and wherein the cutting edge is at least partially curved.

6. The neurosurgical catheter of claim 1, further comprising an elongated macerator positioned with the lumen and rotatable around an axis substantially collinear with a length of the body.

7. The neurosurgical catheter of claim 1, wherein the surface disrupter is a first surface disrupter, and wherein the neurosurgical catheter further comprises:
a second surface disrupter extending from the distal end of the lumen, wherein the second surface disrupter is configured to restrict extension of the first surface disrupter from the distal end of the lumen.

8. The neurosurgical catheter of claim 7, wherein the second surface disrupter includes two or more curved elongated members.

9. The neurosurgical catheter of claim 7, wherein the second surface disrupter is fixed to the distal end of the lumen.

10. A neurosurgical catheter, comprising:
a body at least partially defining a lumen;
a surface disrupter extended or extendable from a distal end of the lumen; and
an elongated macerator positioned with the lumen and rotatable around an axis substantially collinear with a length of the body.

11. The neurosurgical catheter of claim 10, wherein the surface disrupter is configured to be in a collapsed configuration within the lumen and expand into an expanded configuration when extended from the distal end of the lumen, and wherein, in the expanded configuration, the surface disrupter has a diameter greater than a diameter of the lumen.

12. The neurosurgical catheter of claim 10, wherein the surface disrupter includes two or more curved elongated members.

13. The neurosurgical catheter of claim 10, wherein the surface disrupter is substantially shaped as a spheroid or a portion of a spheroid.

14. The neurosurgical catheter of claim 10, wherein the surface disrupter includes an abrasive pattern.

15. The neurosurgical catheter of claim 10, further comprising a driver connected to the surface disrupter and extending proximally through the lumen.

16. The neurosurgical catheter of claim 10, wherein the elongated macerator includes a screw conveyor, and wherein the neurosurgical catheter is configured such that rotating the elongated macerator helps to move material within the lumen proximally along the length of the neurosurgical catheter.

17. The neurosurgical catheter of claim 10, wherein the elongated macerator is sufficiently flexible to bend through angles of a catheterization path.

18. The neurosurgical catheter of claim 10, wherein the elongated macerator is moveable along the length of the neurosurgical catheter.

19. The neurosurgical catheter of claim 10, wherein the surface disrupter is a distal portion of the elongated macerator.

20. The neurosurgical catheter of claim 10, wherein the elongated macerator is configured to transfer rotational or axial movement along at least a portion of the length of the neurosurgical catheter to the surface disrupter.

21. The neurosurgical catheter of claim 10, wherein the elongated macerator includes a spiraling elongated member.

22. The neurosurgical catheter of claim 21, wherein the spiraling elongated member is a wire.

23. A neurosurgical catheter, comprising:
a body at least partially defining a lumen;
a lateral opening extending through a wall of the body and into the lumen at a distal portion of the neurosurgical catheter; and
a surface disrupter movable within the lumen along a length of the neurosurgical catheter such that at least a portion of the surface disrupter slides through the lumen proximate the lateral opening.

24. The neurosurgical catheter of claim 23, wherein the lateral opening extends through a curved wall of the body.

25. The neurosurgical catheter of claim 23, wherein the surface disrupter includes a sharpened edge.

26. A neurosurgical catheter, comprising:
a body at least partially defining a lumen with a distal opening; and
a suction conduit within the lumen, the suction conduit having a main portion and a plug, the plug at least partially defining a plug chamber with a distal opening and a proximal opening into the plug chamber,
wherein the plug is rotatable between (a) a first position in which the distal opening of the body and the distal opening into the plug chamber are substantially aligned and the proximal opening into the plug chamber and the distal opening of the main portion of the suction conduit are not substantially aligned and (b) a second position in which the distal opening of the body and the distal opening into the plug chamber are not substantially aligned and the proximal opening into the plug chamber and the distal opening of the main portion of the suction conduit are substantially aligned.

27. The neurosurgical catheter of claim 26, further comprising a flush conduit within the lumen, wherein the plug is rotatable into (c) a third position in which an opening into the plug chamber is substantially aligned with an opening of the flush conduit and the proximal opening into the plug chamber is substantially aligned with the distal opening of the main portion of the suction conduit or a separate distal opening of the main portion of the suction conduit.

28. A neurosurgical catheterization portal, comprising:
a body configured to be mounted to a surface of a neurosurgical catheterization entry site; and
an adjustable portal having a directional portion at least partially defining a lumen, wherein the lumen is elongated and substantially straight, the neurosurgical catheterization portal has a first configuration in which the adjustable portal is movable relative to the body to angle the directional portion or rotate the directional portion and a second configuration in which the adjustable portal is fixed relative to the body.

29. The neurosurgical catheterization portal of claim 28, wherein the directional portion of the adjustable portal is substantially rigid and has a length between about 10 times and about 50 times a diameter of the lumen.

30. The neurosurgical catheterization portal of claim 28, wherein the body further includes a base configured to be mounted to the surface of the neurosurgical catheterization entry site and a cap separable from the base, and wherein a portion of the adjustable portal is captured between the base and the cap when the neurosurgical catheterization portal is in the second configuration.

31. The neurosurgical catheterization portal of claim 30, wherein the portion of the adjustable portal captured between the base and the cap when the neurosurgical catheterization portal is in the second configuration includes a convex surface, and wherein the cap includes a concave surface adjacent to the convex surface of the adjustable portal when the neurosurgical catheterization portal is in the second configuration.

32. The neurosurgical catheterization portal of claim 30, wherein the base and the cap include interlocking threads, thread recesses, or both allowing the cap to be screwed onto the base.

33. The neurosurgical catheterization portal of claim 30, wherein the base includes two or more mounting tabs having screw-receiving holes, and wherein a living hinge connects each of the mounting tabs to another portion of the base.

34. The neurosurgical catheterization portal of claim 30, wherein the base includes a gasket recess, and wherein the neurosurgical catheterization portal further comprises a gasket configured to be positioned between the gasket recess of the base and the surface of the neurosurgical catheterization entry site.

35. The neurosurgical catheterization portal of claim 30, wherein the base at least partially defines a chamber configured to be adjacent to the surface of the neurosurgical catheterization entry site, and wherein the neurosurgical catheterization portal further comprises a conduit extending between an external portion of the neurosurgical catheterization portal and the chamber.

36. The neurosurgical catheterization portal of claim 35, wherein the conduit is a first conduit, and wherein the neurosurgical catheterization portal further comprises:
a second conduit extending between an external portion of the neurosurgical catheterization portal and the chamber; and
a pump configured to move a flushing fluid into the chamber through the first conduit and out of the chamber through the second conduit.

37. A neurosurgical system, comprising:
a cannula; and
an angle-forming member proximate a distal end of the cannula, wherein the cannula is substantially straight and substantially rigid, and wherein the angle-forming member is configured to transition from a substantially straight configuration while the angle-forming member is advanced through tissue along a substantially straight first portion of a path to an angled configuration when the angle-forming member reaches an end of the substantially straight first portion of the path.

38. The neurosurgical system of claim 37, wherein the angle-forming member has a length between about 3 times and about 10 times its diameter.

39. The neurosurgical system of claim 37, wherein the cannula is a first cannula and the angle-forming member is a first angle-forming member, and wherein the neurosurgical system further comprises:
a second cannula positioned coaxially within or around the first cannula, wherein the second cannula is advanceable along a substantially straight second portion of the path, and wherein the angled configuration of the first angle-forming member corresponds to an angle of the substantially straight second portion of the path relative to the substantially straight first portion of the path.

40. The neurosurgical system of claim 39, wherein the second cannula is substantially flexible.

41. The neurosurgical system of claim 39, further comprising a second angle-forming member proximate a distal end of the second cannula, wherein the second angle-forming member is configured to transition from a substantially straight configuration while the second cannula is advanced through tissue along the substantially straight second portion of the path to an angled configuration when the second angle-forming member reaches an end of the substantially straight second portion of the path.

42. The neurosurgical system of claim 39, further comprising a catheter sized to fit within the cannula and advanceable relative to the cannula so as to extend through a lumen of the angle-forming member when the angle-forming member is in the angled configuration.

43. The neurosurgical system of claim 42, wherein the catheter includes an ultrasound transducer.

44. The neurosurgical system of claim 42, wherein the catheter includes a tip ultrasound transducer proximate a tip of a distal portion of the catheter and two or more radial ultrasound transducers proximate a lateral wall of the distal portion of the catheter.

45. The neurosurgical system of claim 42, further comprising a processing system configured to receive A-mode ultrasound data from an ultrasonography system including an ultrasound transducer within a distal portion of the catheter.

46. A neurosurgical method, comprising:
advancing a cannula having a main portion and an angle-forming member through brain tissue along a first portion of a path to a target area, the main portion being substantially straight, the angle-forming member having a first configuration in which the angle-forming member is substantially straight and a second configuration in which a lumen of the angle-forming member is curved, wherein the first portion of the path is substantially straight, and the angle-forming member is in the first configuration while the cannula is advanced along the first portion of the path;
actuating the angle-forming member to cause the angle-forming member to change from the first configuration to the second configuration after advancing the cannula along the first portion of the path; and
advancing a catheter through the cannula after actuating the angle-forming member such that the catheter extends through a curve of the lumen of the angle-forming member in the second configuration.

47. The neurosurgical method of claim 46, further comprising drilling a hole in bone matter before advancing the cannula through the brain tissue, wherein drilling the hole includes aligning a drilling member with an elongated lumen of a directional portion of a neurosurgical catheterization portal attached to the bone matter, the elongated lumen of the directional portion of the neurosurgical catheterization portal is substantially aligned with the first portion of the path, and the first portion of the path is not substantially perpendicular to a surface around the hole.

48. The neurosurgical method of claim 47, further comprising adjusting a position of the directional portion of the neurosurgical catheterization portal relative to a fixed portion of the neurosurgical catheterization portal, and fixing the directional portion of the neurosurgical catheterization portal to the fixed portion of the neurosurgical catheterization portal in an adjusted position.

49. The neurosurgical method of claim 46, further comprising navigating a distal portion of the catheter using ultrasonography, wherein the distal portion of the catheter includes an ultrasound transducer.

50. The neurosurgical method of claim 49, wherein navigating the distal portion of the catheter using ultrasonography includes navigating the distal portion of the catheter using A-mode ultrasonography.

51. The neurosurgical method of claim 49, wherein navigating the distal portion of the catheter using ultrasonography includes using ultrasonography to monitor a distance between the distal portion of the catheter and an interface between brain tissue and a blood clot.

52. The neurosurgical method of claim 51, wherein using ultrasonography to monitor a distance between the distal portion of the catheter and an interface between brain tissue and a blood clot includes monitoring a distance between a tip ultrasound transducer positioned proximate a tip of the distal portion of the catheter and the interface between brain tissue and the blood clot, monitoring a distance between a first radial ultrasound transducer positioned proximate a lateral wall of the distal portion of the catheter and the interface between brain tissue and the blood clot, and monitoring a distance between a second radial ultrasound transducer positioned proximate the lateral wall of the distal portion of the catheter and the interface between brain tissue and the blood clot.
